SEQUENCE LISTING (66 sequences)

SEQ ID NO: 1 - protein, 18 aa, synthetic construct
  REGION 1..18: note = Hepcidin mimetic
  SITE 1: note = Iovaleric acid conjugation
DTHFPICIFGPRSKGWVC

SEQ ID NO: 2 - protein, 19 aa, synthetic construct
  REGION 1..19: note = Hepcidin mimetic
  SITE 1: note = Isovaleric acid conjugation
DTHFPCIIFGPRSKGWVCK

SEQ ID NO: 3 - protein, 19 aa, synthetic construct
  REGION 1..19: note = Hepcidin mimetic
  SITE 1: note = Isovaleric acid conjugation
DTHFPCIIFEPRSKGWVCK

SEQ ID NO: 4 - protein, 19 aa, synthetic construct
  REGION 1..19: note = Hepcidin mimetic
  SITE 1: note = Isovaleric acid conjugation
DTHFPCIIFGPRSKGWACK

SEQ ID NO: 5 - protein, 20 aa, synthetic construct
  REGION 1..20: note = Hepcidin mimetic
  SITE 1: note = Isovaleric acid conjugation
DTHFPCIIFGPRSKGWVCKK

SEQ ID NO: 6 - protein, 23 aa, synthetic construct
  REGION 1..23: note = Hepcidin mimetic
  SITE 1: note = Isovaleric acid conjugation
DTHFPCIIFVCHRPKGCYRRVCR

SEQ ID NO: 7 - protein, 19 aa, synthetic construct
  REGION 1..19: note = Hepcidin mimetic
  SITE 1: note = Isovaleric acid conjugation
  SITE 8: note = PEG8 conjugation
DTHFPCIKFGPRSKGWVCK

SEQ ID NO: 8 - protein, 19 aa, synthetic construct
  REGION 1..19: note = Hepcidin mimetic
  SITE 1: note = Isovaleric acid conjugation
  SITE 10: note = PEG8 conjugation
DTHFPCIKFKPRSKGWVCK

SEQ ID NO: 9 - protein, 18 aa, synthetic construct
  REGION 1..18: note = Hepcidin mimetic
  SITE 1: note = Isovaleric acid conjugation
  SITE 14: note = PEG8 conjugation
DTHFPICIFGPRSKGWVC

SEQ ID NO: 10 - protein, 18 aa, synthetic construct
  REGION 1..18: note = Hepcidin mimetic
  SITE 1: note = Isovaleric acid conjugation
  SITE 14: note = PEG4 conjugation
DTHFPICIFGPRSKGWVC

SEQ ID NO: 11 - protein, 19 aa, synthetic construct
  REGION 1..19: note = Hepcidin mimetic
  SITE 1: note = Isovaleric acid conjugation
  SITE 19: note = PEG8 conjugation
DTHFPCIKFGPRSKGWVCK

SEQ ID NO: 12 - protein, 19 aa, synthetic construct
  REGION 1..19: note = Hepcidin mimetic
  SITE 1: note = Isovaleric acid conjugation
  SITE 19: note = PEG4 conjugation
DTHFPCIKFKPRSKGWVCK

SEQ ID NO: 13 - protein, 19 aa, synthetic construct
  REGION 1..19: note = Hepcidin mimetic
  SITE 1: note = Isovaleric acid conjugation
  SITE 19: note = PEG2 conjugation
DTHFPCIIFGPRSRGWVCK

SEQ ID NO: 14 - protein, 19 aa, synthetic construct
  REGION 1..19: note = Hepcidin mimetic
  SITE 1: note = Isovaleric acid conjugation
  SITE 8: note = Palmityl conjugation
DTHFPCIKFGPRSKGWVCK

SEQ ID NO: 15 - protein, 19 aa, synthetic construct
  REGION 1..19: note = Hepcidin mimetic
  SITE 1: note = Isovaleric acid conjugation
  SITE 10: note = Palmityl conjugation
DTHFPCIKFKPRSKGWVCK

SEQ ID NO: 16 - protein, 19 aa, synthetic construct
  REGION 1..19: note = Hepcidin mimetic
  SITE 1: note = Isovaleric acid conjugation
  SITE 12: note = Palmityl conjugation
DTHFPCIKFGPKSKGWVCK

SEQ ID NO: 17 - protein, 19 aa, synthetic construct
  REGION 1..19: note = Hepcidin mimetic
  SITE 1: note = Isovaleric acid conjugation
  SITE 14: note = Palmityl conjugation
DTHFPCIKFGPRSKGWVCK

SEQ ID NO: 18 - protein, 19 aa, synthetic construct
  REGION 1..19: note = Hepcidin mimetic
  SITE 1: note = Isovaleric acid conjugation
  SITE 19: note = Palmityl conjugation
DTHFPCIKFGPRSKGWVCK

SEQ ID NO: 19 - protein, 19 aa, synthetic construct
  REGION 1..19: note = Hepcidin mimetic
  SITE 1: note = Isovaleric acid conjugation
  SITE 8: note = PEG3-Palmityl conjugation
DTHFPCIKFGPRSKGWVCK

SEQ ID NO: 20 - protein, 19 aa, synthetic construct
  REGION 1..19: note = Hepcidin mimetic
  SITE 1: note = Isovaleric acid conjugation
  SITE 10: note = PEG3-Palmityl conjugation
DTHFPCIKFKPRSKGWVCK

SEQ ID NO: 21 - protein, 19 aa, synthetic construct
  REGION 1..19: note = Hepcidin mimetic
  SITE 1: note = Isovaleric acid conjugation
  SITE 12: note = PEG3-Palmityl conjugation
DTHFPCIKFGPKSKGWVCK

SEQ ID NO: 22 - protein, 19 aa, synthetic construct
  REGION 1..19: note = Hepcidin mimetic
  SITE 1: note = Isovaleric acid conjugation
  SITE 14: note = PEG3-Palmityl conjugation
DTHFPCIKFGPRSKGWVCK

SEQ ID NO: 23 - protein, 19 aa, synthetic construct
  REGION 1..19: note = Hepcidin mimetic
  SITE 1: note = Isovaleric acid conjugation
  SITE 19: note = PEG3-Palmityl conjugation
DTHFPCIKFGPRSKGWVCK

SEQ ID NO: 24 - protein, 19 aa, synthetic construct
  REGION 1..19: note = Hepcidin mimetic
  SITE 1: note = Isovaleric acid conjugation
  SITE 19: note = PEG8 conjugation
DTHFPCIKFGPRSKGWVCK

SEQ ID NO: 25 - protein, 17 aa, synthetic construct
  REGION 1..17: note = Hepcidin mimetic
  SITE 1: note = Isovaleric acid conjugation
  SITE 8: note = IsoGlu-Palmityl conjugation
DTHFPCIKFEPRSKGCK

SEQ ID NO: 26 - protein, 17 aa, synthetic construct
  REGION 1..17: note = Hepcidin mimetic
  SITE 1: note = Isovaleric acid conjugation
  SITE 10: note = IsoGlu-Palmityl conjugation
DTHFPCIKFKPRSKGCK

SEQ ID NO: 27 - protein, 17 aa, synthetic construct
  REGION 1..17: note = Hepcidin mimetic
  SITE 1: note = Isovaleric acid conjugation
  SITE 12: note = IsoGlu-Palmityl conjugation
DTHFPCIKFEPKSKGCK

SEQ ID NO: 28 - protein, 19 aa, synthetic construct
  REGION 1..19: note = Hepcidin mimetic
  SITE 1: note = Isovaleric acid conjugation
  SITE 12: note = IsoGlu-Palmityl conjugation
DTHFPCIKFEPKSKGWECK

SEQ ID NO: 29 - protein, 17 aa, synthetic construct
  REGION 1..17: note = Hepcidin mimetic
  SITE 1: note = Isovaleric acid conjugation
  SITE 14: note = IsoGlu-Palmityl conjugation
DTHFPCIKFEPRSKGCK

SEQ ID NO: 30 - protein, 17 aa, synthetic construct
  REGION 1..17: note = Hepcidin mimetic
  SITE 1: note = Isovaleric acid conjugation
  SITE 15: note = IsoGlu-Palmityl conjugation
DTHFPCIKFEPRSKKCK

SEQ ID NO: 31 - protein, 17 aa, synthetic construct
  REGION 1..17: note = Hepcidin mimetic
  SITE 1: note = Isovaleric acid conjugation
  SITE 17: note = IsoGlu-Palmityl conjugation
DTHFPCIKFEPRSKKCK

SEQ ID NO: 32 - protein, 17 aa, synthetic construct
  REGION 1..17: note = Hepcidin mimetic
  SITE 1: note = Isovaleric acid conjugation
  SITE 8: note = Dapa-palmityl conjugation
DTHFPCIKFEPRSKGCK

SEQ ID NO: 33 - protein, 16 aa, synthetic construct
  REGION 1..16: note = Hepcidin mimetic
  SITE 1: note = Isovaleric acid conjugation
  SITE 9: note = Dapa-palmityl conjugation
DTHFPCIKFPRSKGCK

SEQ ID NO: 34 - protein, 17 aa, synthetic construct
  REGION 1..17: note = Hepcidin mimetic
  SITE 1: note = Isovaleric acid conjugation
  SITE 12: note = Dapa-palmityl conjugation
DTHFPCIKFEPKSKGCK

SEQ ID NO: 35 - protein, 17 aa, synthetic construct
  REGION 1..17: note = Hepcidin mimetic
  SITE 1: note = Isovaleric acid conjugation
  SITE 14: note = Dapa-palmityl conjugation
DTHFPCIKFEPRSKGCK

SEQ ID NO: 36 - protein, 17 aa, synthetic construct
  REGION 1..17: note = Hepcidin mimetic
  SITE 1: note = Isovaleric acid conjugation
  SITE 15: note = Dapa-palmityl conjugation
DTHFPCIKFEPRSKKCK

SEQ ID NO: 37 - protein, 18 aa, synthetic construct
  REGION 1..18: note = Hepcidin mimetic
  SITE 1: note = Isovaleric acid conjugation
  SITE 17: note = Dapa-palmityl conjugation
DTHFPCIKFEPRSKGCKK

SEQ ID NO: 38 - protein, 17 aa, synthetic construct
  REGION 1..17: note = Hepcidin mimetic
  SITE 1: note = Isovaleric acid conjugation
  SITE 17: note = Dapa-palmityl conjugation
DTHFPCIKFEPRSKGCK

SEQ ID NO: 39 - protein, 17 aa, synthetic construct
  REGION 1..17: note = Hepcidin mimetic
  SITE 1: note = Isovaleric acid conjugation
  SITE 10: note = PEG11-palmityl conjugation
  MOD_RES 15: note = N-methylglycine
DTHFPCIKFKPRSKXCK

SEQ ID NO: 40 - protein, 9 aa, synthetic construct
  REGION 1..9: note = Hepcidin mimetic
  SITE 1: note = Isovaleric acid conjugation
DTHFPCIKF

SEQ ID NO: 41 - protein, 9 aa, synthetic construct
  REGION 1..9: note = Hepcidin mimetic
DTHFPCIKF

SEQ ID NO: 42 - protein, 9 aa, synthetic construct
  REGION 1..9: note = Hepcidin mimetic
  SITE 1: note = Isovaleric acid conjugation
DTHFPCIIF

SEQ ID NO: 43 - protein, 10 aa, synthetic construct
  REGION 1..10: note = Hepcidin mimetic
DTHFPCIIKF

SEQ ID NO: 44 - protein, 9 aa, synthetic construct
  REGION 1..9: note = Hepcidin mimetic
  SITE 1: note = Isovaleric acid conjugation
DTKFPCIIF

SEQ ID NO: 45 - protein, 9 aa, synthetic construct
  REGION 1..9: note = Hepcidin mimetic
DTKFPCIIF

SEQ ID NO: 46 - protein, 17 aa, synthetic construct
  REGION 1..17: note = Hepcidin mimetic
  SITE 1: note = Isovaleric acid conjugation
  SITE 8: note = IsoGlu-Palmityl conjugation
ETHFPCIKFEPRSKGCK

SEQ ID NO: 47 - protein, 18 aa, synthetic construct
  REGION 1..18: note = Hepcidin mimetic
DTHFPICIFGPRSKGWVC

SEQ ID NO: 48 - protein, 19 aa, synthetic construct
  REGION 1..19: note = Hepcidin mimetic
DTHFPCIIFGPRSKGWVCK

SEQ ID NO: 49 - protein, 19 aa, synthetic construct
  REGION 1..19: note = Hepcidin mimetic
DTHFPCIIFEPRSKGWVCK

SEQ ID NO: 50 - protein, 19 aa, synthetic construct
  REGION 1..19: note = Hepcidin mimetic
DTHFPCIIFGPRSKGWACK

SEQ ID NO: 51 - protein, 20 aa, synthetic construct
  REGION 1..20: note = Hepcidin mimetic
DTHFPCIIFGPRSKGWVCKK

SEQ ID NO: 52 - protein, 23 aa, synthetic construct
  REGION 1..23: note = Hepcidin mimetic
DTHFPCIIFVCHRPKGCYRRVCR

SEQ ID NO: 53 - protein, 19 aa, synthetic construct
  REGION 1..19: note = Hepcidin mimetic
DTHFPCIKFGPRSKGWVCK

SEQ ID NO: 54 - protein, 19 aa, synthetic construct
  REGION 1..19: note = Hepcidin mimetic
DTHFPCIKFKPRSKGWVCK

SEQ ID NO: 55 - protein, 19 aa, synthetic construct
  REGION 1..19: note = Hepcidin mimetic
DTHFPCIIFGPRSRGWVCK

SEQ ID NO: 56 - protein, 19 aa, synthetic construct
  REGION 1..19: note = Hepcidin mimetic
DTHFPCIKFGPKSKGWVCK

SEQ ID NO: 57 - protein, 17 aa, synthetic construct
  REGION 1..17: note = Hepcidin mimetic
DTHFPCIKFEPRSKGCK

SEQ ID NO: 58 - protein, 19 aa, synthetic construct
  REGION 1..19: note = Hepcidin mimetic
DTHFPCIKFEPKSKGWECK

SEQ ID NO: 59 - protein, 17 aa, synthetic construct
  REGION 1..17: note = Hepcidin mimetic
DTHFPCIKFEPRSKKCK

SEQ ID NO: 60 - protein, 18 aa, synthetic construct
  REGION 1..18: note = Hepcidin mimetic
DTHFPCIKFEPRSKGCKK

SEQ ID NO: 61 - protein, 17 aa, synthetic construct
  REGION 1..17: note = Hepcidin mimetic
DTHFPCIKFKPRSKGCK

SEQ ID NO: 62 - protein, 17 aa, synthetic construct
  REGION 1..17: note = Hepcidin mimetic
DTHFPCIKFEPKSKGCK

SEQ ID NO: 63 - protein, 9 aa, synthetic construct
  REGION 1..9: note = Hepcidin mimetic
DTHFPCIKF

SEQ ID NO: 64 - protein, 9 aa, synthetic construct
  REGION 1..9: note = Hepcidin mimetic
DTHFPCIIF

SEQ ID NO: 65 - protein, 9 aa, synthetic construct
  REGION 1..9: note = Hepcidin mimetic
DTKFPCIIF

SEQ ID NO: 66 - protein, 25 aa, Homo sapiens
  DISULFID 7..23: note = Dusulfide linkage between cysteine residues 7 and 23
  DISULFID 10..13: note = Dusulfide linkage between cysteine residues 10 and 13
  DISULFID 11..19: note = Dusulfide linkage between cysteine residues 11 and 19
  DISULFID 14..22: note = Dusulfide linkage between cysteine residues 14 and 22
DTHFPICIFCCGCCHRSKCGMCCKT

(19) United States
(12) Patent Application Publication
(10) Pub. No.: US 2024/0016895 A1
(43) Pub. Date: Jan. 18, 2024

(54) HEPCIDIN MIMETICS FOR TREATMENT OF SICKLE CELL DISEASE

(71) Applicant: Protagonist Therapeutics, Inc., Newark, CA (US)

(72) Inventors: David Y. LIU, Milpitas, CA (US); Gregory Thomas BOURNE, Moggill (AU); Roopa TARANATH, Cupertino, CA (US); Li ZHAO, Mountain View, CA (US)

(21) Appl. No.: 18/355,992

(22) Filed: Jul. 20, 2023

Related U.S. Application Data
(63) Continuation of application No. PCT/US2023/067988, filed on Jun. 6, 2023.
(60) Provisional application No. 63/349,908, filed on Jun. 7, 2022.

(51) Int. Cl. A61K 38/22; A61P 7/06

(52) U.S. Cl. CPC A61K 38/22; A61P 7/06

(57) ABSTRACT

The disclosure provides compositions and methods for the treatment and/or prevention of sickle cell disease.

### CROSS REFERENCES TO RELATED APPLICATIONS

This application is a continuation of International PCT Patent Application No. PCT/US2023/067988, which was filed on Jun. 6, 2023, now pending, which claims priority to U.S. Provisional Application No. 63/349,908, filed Jun. 7, 2022, each of which is incorporated herein in their entirety for all purposes.

### STATEMENT REGARDING SEQUENCE LISTING

The Sequence Listing XML associated with this application is provided in XML file format and is hereby incorporated by reference into the specification. The name of the XML file containing the Sequence Listing XML is PRTH_080_01WO_ST26.xml. The XML file is 114,330 bytes, and created on Jul. 19, 2023, and is being submitted electronically via USPTO Patent Center.

### FIELD OF THE INVENTION

The present disclosure relates, inter alia, to compositions and methods for the treatment and/or prevention of sickle cell disease.

### BACKGROUND

Sickle cell disease (SCD) is a group of hereditary blood disorders. SCD is characterized by abnormal hemoglobin, which results in red blood cells that assume a distorted, rigid, sickle shape. Healthy red blood cells are round, and they move through small blood vessels to carry oxygen to all parts of the body. The sickle-shaped red blood cells are prone to intravascular hemolysis and intermittent blood flow occlusion. When these sickle cells travel through small blood vessels, they get stuck and clog the blood flow. This can result in episode of severe pain and ischemia-reperfusion injury of the organs, like kidney failure, liver pathology, stroke, infection due to splenic infarction, and other complications.

Different forms of SCD include the homozygous sickle-cell anemia (HbSS), the heterozygous sickle-cell trait (HbAC), and the hemoglobin SC disease (HbSC), sickle-hemoglobin D (HbSD), sickle-hemoglobin E (HbSE), sickle-hemoglobin O (HbSO), sickle-beta-plus-thalassemia (HbSβ+thalassemia), sickle-beta-zero-thalassemia (HbSβ₀ thalassemia).

There is currently a large, unmet medical need for safe and effective therapies for the treatment of sickle cell disease and its complications. The present disclosure meets this and other needs.

### SUMMARY OF THE INVENTION

The present disclosure is related to the use of hepcidin mimetics or peptides to treat sickle cell diseases.

In one aspect, the present disclosure provides a method for treating sickle cell disease in a subject, the method comprising administering to the subject an effective amount of a hepcidin mimetic as disclosed herein or a pharmaceutically acceptable salt thereof or a composition comprising a hepcidin mimetic as disclosed herein or a pharmaceutically acceptable salt thereof or a pharmaceutical composition comprising a hepcidin mimetic or a pharmaceutically acceptable salt thereof and a pharmaceutically acceptable excipient or carrier. In some embodiments, the hepcidin mimetics is a peptide. The subtypes or genotypes of sickle cell disease treatable with a hepcidin mimetic as disclosed herein include, but are not limited to, sickle cell anemia (HbSS), HbSβ0 thalassemia, HbSβ+thalassemia, and hemoglobin SC disease (HbSC).

In some embodiments, the present disclosure provides a method for treating sickle cell disease or a subtype or genotype of sickle cell disease in a subject, the method comprising administering to the subject an effective amount of a hepcidin mimetic, which is a peptide comprising or having Formula (I):

R1-X—Y—R2 (I)
or a pharmaceutically acceptable salt or solvate thereof,
wherein R1 is hydrogen, a C1-C6 alkyl, a C6-C12 aryl, a C1-C20 alkanoyl, or pGlu;
R2 is NH₂ or OH;
X is an amino acid sequence of Formula II:

X1-X2-X3-X4-X5-X6-X7-X8-X9-X10 (II)
wherein
X1 is Asp, Ala, Ida, pGlu, bhAsp, Leu, D-Asp, or absent;
X2 is Thr, Ala, or D-Thr;
X3 is His, Lys, D-His, or Lys;
X4 is Phe, Ala, Dpa, or D-Phe;
X5 is Pro, Gly, Arg, Lys, Ala, D-Pro, or bhPro;
X6 is Ile, Cys, Arg, Lys, D-Ile, or D-Cys;
X7 is Cys, Ile, Leu, Val, Phe, D-Ile, or D-Cys;
X8 is Ile, Arg, Phe, Gln, Lys, Glu, Val, Leu, or D-Ile;
X9 is Phe or bhPhe; and
X10 is Lys, Phe, or absent;
wherein if Y is absent, X7 is Ile; and
Y is an amino acid sequence of Formula III:

Y1-Y2-Y3-Y4-Y5-Y6-Y7-Y8-Y9-Y10-Y11-Y12-Y13-Y14-Y15 (III)
wherein
Y1 is Gly, Cys, Ala, Phe, Pro, Glu, Lys, D-Pro, Val, Ser, or absent;
Y2 is Pro, Ala, Cys, Gly, or absent;
Y3 is Arg, Lys, Pro, Gly, His, Ala, Trp, or absent;
Y4 is Ser, Arg, Gly, Trp, Ala, His, Tyr, or absent;
Y5 is Lys, Met, Arg, Ala, or absent;
Y6 is Gly, Ser, Lys, Ile, Ala, Pro, Val, or absent;
Y7 is Trp, Lys, Gly, Ala, Ile, Val, or absent;
Y8 is Val, Thr, Gly, Cys, Met, Tyr, Ala, Glu, Lys, Asp, Arg, or absent;
Y9 is Cys, Tyr, or absent;
Y10 is Met, Lys, Arg, Tyr, or absent;
Y11 is Arg, Met, Cys, Lys, or absent;
Y12 is Arg, Lys, Ala or absent;
Y13 is Arg, Cys, Lys, Val or absent;
Y14 is Arg, Lys, Pro, Cys, Thr or absent; and
Y15 is Thr, Arg or absent;
wherein the peptide of Formula I is optionally PEGylated on R1, X, or Y;
wherein a side chain of an amino acid of the peptide is optionally conjugated to a lipophilic substituent or polymeric moiety;
wherein the peptide of Formula I optionally has a disulfide bond formed between the thiol groups of two cysteine residues; and
wherein Ida is iminodiacetic acid, pGlu is pyroglutamic acid, bhAsp is β-homoaspartic acid, and bhPro is β-homoproline. In some embodiments of compounds of formula (I), a disulfide bond formed between the thiol groups of two cysteine residues. In one embodiment, the hepcidin mimetic or pharmaceutically acceptable salt thereof or pharmaceutical composition is administered subcutaneously.

In some embodiments, the hepcidin mimetic comprises or consists of a peptide of any one of Formulas I-VIII as disclosed herein. In certain embodiments, the peptide comprises or consists of one of the following sequences or structures:

(Compound 1; SEQ ID NO: 1))
Isovaleric acid-DTHFPICIFGPRSKGWVC-NH₂;
(Compound 2; SEQ ID NO: 2)
Isovaleric acid-DTHFPCIIFGPRSKGWVCK-NH₂;
(Compound 3; SEQ ID NO: 3)
Isovaleric acid-DTHFPCIIFEPRSKGWVCK-NH₂;
(Compound 4; SEQ ID NO: 4)
Isovaleric acid-DTHFPCIIFGPRSKGWACK-NH₂;
(Compound 5; SEQ ID NO: 5)
Isovaleric acid-DTHFPCIIFGPRSKGWVCKK-NH₂;
(Compound 6; SEQ ID NO: 6)
Isovaleric acid-DTHFPCIIFVCHRPKGCYRRVCR-NH₂;
(Compound 7; SEQ ID NO: 7)
Isovaleric acid-DTHFPCI(K(PEG8))FGPRSKGWVCK-NH₂;
(Compound 8; SEQ ID NO: 8)
Isovaleric acid-DTHFPCIKF(K(PEG8))PRSKGWVCK-NH₂;
(Compound 9; SEQ ID NO: 9)
Isovaleric acid-DTHFPICIFGPRS(K(PEG8))GWVC-NH₂;
(Compound 10; SEQ ID NO: 10)
Isovaleric acid-DTHFPICIFGPRS(K(PEG4))GWVC-NH₂;
(Compound 11; SEQ ID NO: 11)
Isovaleric acid-DTHFPCIIFGPRSRGWVC(K(PEG8))-NH₂;
(Compound 12; SEQ ID NO: 12)
Isovaleric acid-DTHFPCIIFGPRSRGWVC(K(PEG4))-NH₂;
(Compound 13; SEQ ID NO: 13)
Isovaleric acid-DTHFPCIIFGPRSRGWVC(K(PEG2))-NH₂;
(Compound 14; SEQ ID NO: 14)
Isovaleric acid-DTHFPCI(K(Palm))FGPRSKGWVCK-NH₂;
(Compound 15; SEQ ID NO: 15)
Isovaleric acid-DTHFPCIKF)K(Palm))PRSKGWVCK-NH₂;
(Compound 16; SEQ ID NO: 16)
Isovaleric acid-DTHFPCIKFGP(K(Palm))SKGWVCK-NH₂;
(Compound 17; SEQ ID NO: 17)
Isovaleric acid-DTHFPCIKFGPRS(K(Palm))GWVCK-NH₂;
(Compound 18; SEQ ID NO: 18)
Isovaleric acid-DTHFPCIKFGPRSKGWVC(K(Palm))NH₂;
(Compound 19; SEQ ID NO: 19)
Isovaleric acid-DTHFPCI(K(PEG3-Palm))FGPRSKGWVCK-NH₂;
(Compound 20; SEQ ID NO: 20)
Isovaleric acid-DTHFPCIKF(K(PEG3-Palm))PRSKGWVCK-NH₂;
(Compound 21; SEQ ID NO: 21)
Isovaleric acid-DTHFPCIKFGP(K(PEG3-Palm))SKGWVCK-NH₂;
(Compound 22; SEQ ID NO: 22)
Isovaleric acid-DTHFPCIKFGPRS(K(PEG3-Palm))GWVCK-NH₂;
(Compound 23; SEQ ID NO: 23)
Isovaleric acid-DTHFPCIKFGPRSKGWVC(K(PEG3-Palm))-NH₂;
(Compound 24; SEQ ID NO: 24)
Isovaleric acid-DTHFPCIKFGPRSKGWVC(K(PEG8))-NH₂;
(Compound 25; SEQ ID NO: 25)
Isovaleric acid-DTHFPCI(K(isoGlu-Palm))FEPRSKGCK-NH₂;
(Compound 26; SEQ ID NO: 26)
Isovaleric acid-DTHFPCIKF-K(isoGlu-Palm)-PRSKGCK-NH₂;
(Compound 27; SEQ ID NO: 27)
Isovaleric acid-DTHFPCIKFEP(K(isoGlu-Palm))SKGCK-NH₂;
(Compound 28; SEQ ID NO: 28)
Isovaleric acid-DTHFPCIKFEP(K(isoGlu-Palm))SKGWECK-NH₂;
(Compound 29; SEQ ID NO: 29)
Isovaleric acid-DTHFPCIKFEPRS(K(isoGlu-Palm))GCK-NH₂;
(Compound 30; SEQ ID NO: 30)
Isovaleric acid-DTHFPCIKFEPRSK(K(isoGlu-Palm))CK-NH₂;
(Compound 31; SEQ ID NO: 31)
Isovaleric acid-DTHFPCIKFEPRSKGCK(K(isoGlu-Palm))-NH₂;
(Compound 32; SEQ ID NO: 32)
Isovaleric acid-DTHFPCI-K(Dapa-Palm)-FEPRSKGCK-NH₂;
(Compound 33; ; SEQ ID NO: 33)
Isovaleric acid-DTHFPCIK(F(Dapa-Palm))PRSKGCK-NH₂;
(Compound 34; SEQ ID NO: 34)
Isovaleric acid-DTHFPCIKFEP(K(Dapa-Palm))SKGCK-NH₂;
(Compound 35; SEQ ID NO: 35)
Isovaleric acid-DTHFPCIKFEPRS(K(Dapa-Palm))GCK-NH₂;
(Compound 36; SEQ ID NO: 36)
Isovaleric acid-DTHFPCIKFEPRSK(K(Dapa-Palm))CK-NH₂;
(Compound 37; SEQ ID NO: 37)
Isovaleric acid-DTHFPCIKFEPRSKGC(K(Dapa-Palm))K-NH₂;
(Compound 38; SEQ ID NO: 38)
Isovaleric acid-DTHFPCIKFEPRSKGC(K(Dapa-Palm))-NH₂;
(Compound 39; SEQ ID NO: 39)
Isovaleric acid-DTHFPCIKF(K(PEG11-Palm))PRSK[Sar]CK-NH₂;
(Compound 40; SEQ ID NO: 40)
Isolvaleric acid-DTHFPCIKF-NH₂;
(Compound 41; SEQ ID NO: 41)
Hy-DTHFPCIKF-NH₂;
(Compound 42; SEQ ID NO: 42)
Isolvaleric acid-DTHFPCIIF-NH₂;
(Compound 43; SEQ ID NO: 43)
Hy-DTHFPCIIKF-NH₂;
(Compound 44; SEQ ID NO: 44)
Isovaleric acid-DTKFPCIIF-NH₂;
(Compound 45; SEQ ID NO: 45)
Hy-DTKFPCIIF-NH₂;
or
(Compound 46; SEQ ID NO: 46)
Isovaleric_Acid-ETHFPCI(K(IsoGlu_Palm))FEPRSKGCK-NH₂

wherein the thiol groups of two cysteine residues in each of compounds 1-40 and 46 are optionally taken together to form a disulfide bond.

### BRIEF DESCRIPTION OF THE DRAWINGS

FIG. 1A and FIG. 1B provide graphs showing compound 46 reduces spleen size in Townes SCD mice. FIG. 1A: spleen weights. FIG. 1B: spleen body weight ratio. Individual values and mean±SD are shown, statistical analysis was performed by comparing all treatment groups to the HbSS vehicle group using one-way ANOVA with Dunnett's multiple comparison test, *p<0.05, **p<0.01, ***p<0.001, ****p<0.0001, n=6-10 mice per group.

FIG. 2A and FIG. 2B provide graphs showing compound 46 reduces liver size in Townes SCD mice. FIG. 2A: liver weights. FIG. 1B: liver body weight ratio. Individual values and mean±SD are shown, statistical analysis was performed by comparing all treatment groups to the HbSS vehicle group using one-way ANOVA with Dunnett's multiple comparison test, * p<0.05, **p<0.01, ***p<0.001, ****p<0.0001, n=6-10 mice per group.

FIG. 3A and FIG. 3B provide graphs showing red blood cell (RBC) and hemoglobin indices in Townes mice treated for 4 weeks with Compound 46 or vehicle. FIG. 3A: RBC. FIG. 3B: hemoglobin. Individual values and mean±SD are shown, statistical analysis was performed by comparing all treatment groups to the HbSS vehicle group using one-way ANOVA with Dunnett's multiple comparison test, *p<0.05, **p<0.01, ***p<0.001, ****p<0.0001, n=6-10 mice per group.

FIG. 4A and FIG. 4B provide graphs showing reticulocyte counts in Townes mice treated for 4 weeks with Compound 46 or vehicle. FIG. 4A: absolute reticulocyte count changes. FIG. 4B: percentage reticulocyte changes. Individual values and mean±SD are shown, statistical analysis was performed by comparing all treatment groups to the HbSS vehicle group using one-way ANOVA with Dunnett's multiple comparison test, *p<0.05, ** p<0.01, ***p<0.001, ****p<0.0001, n=6-10 mice per group.

FIG. 5A and FIG. 5B provide graphs showing hematocrit (HCT) and MCV changes in Townes mice treated for 4 weeks with Compound 46 or vehicle. FIG. 5A: HCT changes in percentage. FIG. 5B: MCV changes in volume. Individual values and mean±SD are shown, statistical analysis was performed by comparing all treatment groups to the HbSS vehicle group using one-way ANOVA with Dunnett's multiple comparison test, *p<0.05, **p<0.01, *** p<0.001, ****p<0.0001, n=6-10 mice per group.

FIG. 6A and FIG. 6B provide graphs showing mean corpuscular hemoglobin (MCH) hematocrit (HCT) and mean corpuscular hemoglobin concentration (MCHC) changes in Townes mice treated for 4 weeks with Compound 46 or vehicle. FIG. 6A: MCH changes in mass. FIG. 6B: MCHC changes in concentration. Individual values and mean±SD are shown, statistical analysis was performed by comparing all treatment groups to the HbSS vehicle group using one-way ANOVA with Dunnett's multiple comparison test, *p<0.05, **p<0.01, *** p<0.001, ****p<0.0001, n=6-10 mice per group.

FIG. 7A and FIG. 7B provide graphs showing compound 46 alleviated elevated white blood cell (WBC) and lymphocytes counts in Townes SCD mice. FIG. 7A: changes in WBC counts. FIG. 7B: changes in lymphocyte counts. Individual values and mean±SD are shown, statistical analysis was performed by comparing all treatment groups to the HbSS vehicle group using one-way ANOVA with Dunnett's multiple comparison test, *p<0.05, **p<0.01, *** p<0.001, ****p<0.0001, n=6-10 mice per group.

FIG. 8A and FIG. 8B provide graphs showing compound 46 alleviated elevated neutrophil and monocyte counts in Townes SCD mice. FIG. 8A: changes in neutrophil counts. FIG. 8B: changes in monocyte counts. Individual values and mean±SD are shown, statistical analysis was performed by comparing all treatment groups to the HbSS vehicle group using one-way ANOVA with Dunnett's multiple comparison test, *p<0.05, **p<0.01, ***p<0.001, ****p<0.0001, n=6-10 mice per group.

FIG. 9A and FIG. 9B provide graphs showing effect of compound 46 on serum red blood cell (RBC) hemolysis biomarkers total bilirubin and lactate dehydrogenase (LDH) from Townes mice. FIG. 9A: total bilirubin. FIG. 9B: LDH. Individual values and mean±SD are shown, statistical analysis was performed by comparing all treatment groups to the HbSS vehicle group using one way ANOVA with Dunnett's multiple comparison test, *p<0.05, ** p<0.01, ***p<0.001, ****p<0.0001, n=6-10 mice per group

### DETAILED DESCRIPTION

The present disclosure relates to compounds, compositions, and methods for treating sickle cell diseases. In some embodiments, the disclosure provides methods using compounds as disclosed herein, or a pharmaceutically acceptable salt thereof, or a composition comprising the compound or pharmaceutically acceptable salt thereof as disclosed herein to treat sickle cell diseases.

### Definitions and Nomenclature

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

As used herein, the following terms have the meanings ascribed to them unless specified otherwise.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

“About” when referring to a value includes the stated value+/−10% of the stated value. For example, about 50% includes a range of from 45% to 55%, while about 20 molar equivalents includes a range of from 18 to 22 molar equivalents, and about 10 mg includes a range of from 9 mg to 11 mg. Accordingly, when referring to a range, “about” refers to each of the stated values+/−10% of the stated value of each end of the range. For instance, a ratio of from about 1 to about 3 (weight/weight) includes a range of from 0.9 to 3.3.

The term “including” is used to mean “including but not limited to.” “Including” and “including but not limited to” are used interchangeably.

The terms “patient,” “subject,” and “individual” may be used interchangeably and refer to either a human or a non-human animal. These terms include mammals such as humans, primates, livestock animals (e.g., bovines, porcines), companion animals (e.g., canines, felines) and rodents (e.g., mice and rats). The term “mammal” refers to any mammalian species such as a human, mouse, rat, dog, cat, hamster, guinea pig, rabbit, livestock, and the like.

The term “peptide,” as used herein, refers broadly to a sequence of two or more amino acids joined together by peptide bonds. It should be understood that this term does not connote a specific length of a polymer of amino acids, nor is it intended to imply or distinguish whether the polypeptide is produced using recombinant techniques, chemical or enzymatic synthesis, or is naturally occurring.

The term “hepcidin mimetic,” as used herein, refers broadly to peptide monomers and peptide dimers comprising one or more structural features and/or functional activities in common with hepcidin, or a functional region thereof. In certain embodiments, a hepcidin mimetic includes peptides sharing substantial amino acid sequence identity with hepcidin, e.g., peptides that comprise one or more amino acid insertions, deletions, or substitutions as compared to a wild-type hepcidin, e.g., human hepcidin, amino acid sequence. In certain embodiments, a hepcidin mimetic comprises one or more additional modification, such as, e.g., conjugation to another compound. Encompassed by the term “hepcidin mimetic” is any peptide monomer or peptide dimer disclosed herein. In some embodiments, a hepcidin mimetic has one or more functional activities of hepcidin.

The term “amino acid” or “any amino acid” as used here refers to any and all amino acids, including naturally occurring amino acids (e.g., a-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The “non-standard,” natural amino acids are pyrrolysine (found in methanogenic organisms and other eukaryotes), selenocysteine (present in many noneukaryotes as well as most eukaryotes), and N-formylmethionine (encoded by the start codon AUG in bacteria, mitochondria and chloroplasts). “Unnatural” or “non-natural” amino acids are non-proteinogenic amino acids (i.e., those not naturally encoded or found in the genetic code) that either occur naturally or are chemically synthesized. Over 140 natural amino acids are known and thousands of more combinations are possible. Examples of “unnatural” amino acids include β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid.

As is clear to the skilled artisan, the peptide sequences disclosed herein are shown proceeding from left to right, with the left end of the sequence being the N-terminus of the peptide and the right end of the sequence being the C-terminus of the peptide. Among sequences disclosed herein are sequences incorporating a “Hy-” moiety at the amino terminus (N-terminus) of the sequence, and either an “—OH” moiety or an “—NH₂” moiety at the carboxy terminus (C-terminus) of the sequence. In such cases, and unless otherwise indicated, a “Hy-moiety” at the N-terminus of the sequence in question indicates a hydrogen atom, corresponding to the presence of a free primary or secondary amino group at the N-terminus, while an “—OH” or an “—NH₂” moiety at the C-terminus of the sequence indicates a hydroxy group or an amino group, corresponding to the presence of an amido (CONH₂) group at the C-terminus, respectively. In each sequence of the invention, a C-terminal “—OH” moiety may be substituted for a C-terminal “—NH₂” moiety, and vice-versa. It is further understood that the moiety at the amino terminus or carboxy terminus may be a bond, e.g., a covalent bond, particularly in situations where the amino terminus or carboxy terminus is bound to a linker or to another chemical moiety, e.g., a PEG moiety.

The term “NH₂,” as used herein, refers to the free amino group present at the amino terminus of a polypeptide. The term “OH,” as used herein, refers to the free carboxy group present at the carboxy terminus of a peptide. Further, the term “Ac,” as used herein, refers to Acetyl protection through acylation of the C- or N-terminus of a polypeptide.

The term “carboxy,” as used herein, refers to —CO₂H.

For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader. Some abbreviations useful in describing the invention are defined below in the following Table IA.

TABLE 1A
Abbreviations of Non-Natural Amino Acids and Chemical Moieties

| Abbreviation | Definition |
|---|---|
| DIG | Diglycolic acid |
| Dapa | Diaminopropionic acid |
| Daba | Diaminobutyric acid |
| Pen | Penicillamine |
| Sarc or Sar | Sarcosine |
| Cit | Citroline |
| Cav | Cavanine |
| NMe-Arg | N-Methyl-Arginine |
| NMe-Trp | N-Methyl-Tryptophan |
| NMe-Phe | N-Methyl-Phenylalanine |
| Ac- | Acetyl |
| 2-Nal | 2-Napthylalanine |
| 1-Nal | 1-Napthylalanine |
| Bip | Biphenylalanine |
| βAla | beta-Alanine |
| Aib | 2-aminoisobutyric acid |
| Azt | azetidine-2-carboxylic acid |
| Tic | (3S)-1,2,3,4-Tetrahydroisoquinoline-hydroxy-3-carboxylic |
| | acid |
| Phe(OMe) | Tyrosine (4-Methyl) |
| N-MeLys | N-Methyl-Lysine |
| N-MeLys(Ac) | N-e-Acetyl-D-lysine |
| Dpa | β,β diphenylalanine |
| NH₂ | Free Amine |
| CONH₂ | Amide |
| COOH | Acid |
| Phe(4-F) | 4-Fluoro-Phenylalanine |
| PEG3 | NH₂CH₂CH₂(OCH₂CH₂)₃CH₂CH₂CO₂H |
| m-PEG3 | CH₃OCH₂CH₂(OCH₂CH₂)₂CH₂CH₂CO₂H |
| m-PEG4 | CH₃OCH₂CH₂(OCH₂CH₂)₃CH₂CH₂CO₂H |
| m-PEG8 | CH₃OCH₂CH₂(OCH₂CH₂)₇CH₂CH₂CO₂H |
| PEG11 | O-(2-aminoethy1)-O′-(2-carboxyethyl)-undecaethyleneglycol |
| | NH₂CH₂CH₂(OCH₂CH₂)₁₁CH₂CH₂CO₂H |
| PEG13 | Bifunctional PEG linker with 13 PolyEthylene Glycol units |
| PEG25 | Bifunctional PEG linker with 25 PolyEthylene Glycol units |
| PEG1K | Bifunctional PEG linker with PolyEthylene Glycol Mol wt of |
| | 1000 Da |
| PEG2K | Bifunctional PEG linker with PolyEthylene Glycol Mol wt of |
| | 2000 Da |
| PEG3.4K | Bifunctional PEG linker with PolyEthylene Glycol Mol wt of |
| | 3400 Da |
| PEG5K | Bifunctional PEG linker with PolyEthylene Glycol Mol wt of |
| | 5000 Da |
| IDA or Ida | Iminodiacetic acid |
| IDA-Palm | (Palmityl)-Iminodiacetic acid |
| hPhe | homoPhenylalanine |
| Ahx | Aminohexanoic acid |
| DIG-OH | Glycolic monoacid |
| Triazine | Amino propyl Triazine di-acid |
| Boc-Triazine | Boc-Triazine di-acid |
| Trifluorobutyric acid | 4,4,4-Trifluorobutyric acid |
| 2-Methylltrifluorobutyric acid | 2-methyl-4,4,4-Butyric acid |
| Trifluorpentanoic acid | 5,5,5-Trifluoropentanoic acid |
| 1,4-Phenylenediacetic acid | para-Phenylenediacetic acid |
| 1,3-Phenylenediacetic acid | meta-Phenylenediacetic acid |
| DTT | Dithiothreotol |
| Nle | Norleucine |
| βhTrp or bhTrp | β-homoTryptophane |
| βhPhe or bhPhe | β-homophenylalanine |
| Phe(4-CF₃) | 4-TrifluoromethylPhenylalanine |
| βGlu or bGlu | β-Glutamic acid |
| βhGlu or bhGlu | β-homoglutamic acid |
| 2-2-Indane | 2-Aminoindane-2-carboxylic acid |
| 1-1-Indane | 1-Aminoindane-1-carboxylic acid |
| hCha | homocyclohexylalanine |
| Cyclobutyl | Cyclobutylalanine |
| hLeu | Homoleucine |
| Gla | γ-Carboxy-glutamic acid |
| Aep | 3-(2-aminoethoxy)propanoic acid |
| Aea | (2-aminoethoxy)acetic acid |
| IsoGlu-octanoic acid | octanoyl-γ-Glu |
| K-octanoic acid | octanoyl-ε-Lys |
| Dapa(Palm) | Hexadecanoyl-β-Diaminopropionic acid |
| IsoGlu-Palm | hexadecanoyl-γ-Glu |
| C-StBu | S-tert-butylthio-cysteine |
| C-tBu | S-tert-butyl-cysteine |
| Dapa(AcBr) | NY-(bromoacetyl)-2,3-diaminopropionic acid |
| Tle | tert-Leucine |
| Phg | phenylglycine |
| Oic | octahydroindole-2-carboxylic acid |
| Chg | α-cyclohexylglycine |
| GP-(Hyp) | Gly-Pro-HydroxyPro |
| Inp | isonipecotic acid |
| Amc | 4-(aminomethyl)cyclohexane carboxylic acid |
| Betaine | (CH₃)₃NCH₂CH₂CO2H |
| D-Npc | Isonipecotic acid |
| Npc | Nipecotic acid |

Throughout the present specification, unless naturally occurring amino acids are referred to by their full name (e.g., alanine, arginine, etc.), they are designated by their conventional three-letter or single-letter abbreviations (e.g., Ala or A for alanine, Arg or R for arginine, etc.). In the case of less common or non-naturally occurring amino acids, unless they are referred to by their full name (e.g. sarcosine, ornithine, etc.), frequently employed three- or four-character codes are employed for residues thereof, including, Sar or Sarc (sarcosine, i.e., N-methylglycine), Aib (α-aminoisobutyric acid), Daba (2,4-diaminobutanoic acid), Dapa (2,3-diaminopropanoic acid), γ-Glu (γ-glutamic acid), pGlu (pyroglutamic acid), Gaba (γ-aminobutanoic acid), β-Pro (pyrrolidine-3-carboxylic acid), 8Ado (8-amino-3,6-dioxaoctanoic acid), Abu (4-aminobutyric acid), bhPro (β-homo-proline), bhPhe (β-homo-L-phenylalanine), bhAsp (β-homo-aspartic acid]), Dpa (β,β diphenylalanine), Ida (Iminodiacetic acid), hCys (homocysteine), and bhDpa (β-homo-β,β-diphenylalanine).

Furthermore, R1 can in all sequences be substituted with isovaleric acids or equivalent. In some embodiments, wherein a peptide of the present invention is conjugated to an acidic compound such as, e.g., isovaleric acid, isobutyric acid, valeric acid, and the like, the presence of such a conjugation is referenced in the acid form. So, for example, but not to be limited in any way, instead of indicating a conjugation of isovaleric acid to a peptide by referencing isovaleroyl, in some embodiments, the present application may reference such a conjugation as isovaleric acid.

The term “L-amino acid,” as used herein, refers to the “L” isomeric form of a peptide, and conversely the term “D-amino acid” refers to the “D” isomeric form of a peptide. In certain embodiments, the amino acid residues described herein are in the “L” isomeric form, however, residues in the “D” isomeric form can be substituted for any L-amino acid residue, as long as the desired functional is retained by the peptide.

Unless otherwise indicated, reference is made to the L-isomeric forms of the natural and unnatural amino acids in question possessing a chiral center. Where appropriate, the D-isomeric form of an amino acid is indicated in the conventional manner by the prefix “D” before the conventional three-letter code (e.g., Dasp, (D)Asp or D-Asp; Dphe, (D)Phe or D-Phe).

The term “dimer,” as used herein, refers broadly to a peptide comprising two or more monomer subunits. Certain dimers comprise two DRPs. Dimers of the present invention include homodimers and heterodimers. A monomer subunit of a dimer may be linked at its C- or N-terminus, or it may be linked via internal amino acid residues. Each monomer subunit of a dimer may be linked through the same site, or each may be linked through a different site (e.g., C-terminus, N-terminus, or internal site).

As used herein, in the context of certain peptide sequences disclosed herein, parentheticals, e.g., (__), represent side chain conjugations and brackets, e.g., [_], represent unnatural amino acid substitutions or amino acids and conjugated side chains. Generally, where a linker is shown at the N-terminus of a peptide sequence, it indicates that the peptide is dimerized with another peptide, wherein the linker is attached to the N-terminus of the two peptides. Generally, where a linker is shown at the C-terminus of a peptide sequence or structure, it indicates that the peptide is dimerized with another peptide, wherein the linker is attached to the C-terminus of the two peptides.

The term “cyclized,” as used herein, refers to a reaction in which one part of a polypeptide molecule becomes linked to another part of the polypeptide molecule to form a closed ring, such as by forming a disulfide bridge or other similar bond.

The term “subunit,” as used herein, refers to one of a pair of polypeptide monomers that are joined to form a dimer peptide composition.

The term “linker moiety,” as used herein, refers broadly to a chemical structure that is capable of linking or joining together two peptide monomer subunits to form a dimer.

The term “solvate” in the context of the present invention refers to a complex of defined stoichiometry formed between a solute (e.g., a hepcidin mimetic or pharmaceutically acceptable salt thereof according to the invention) and a solvent. The solvent in this connection may, for example, be water, ethanol or another pharmaceutically acceptable, typically small-molecular organic species, such as, but not limited to, acetic acid or lactic acid. When the solvent in question is water, such a solvate is normally referred to as a hydrate.

The term “pharmaceutically acceptable salt,” as used herein, represents salts or zwitterionic forms of the peptides or compounds of the present invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate (isethionate), lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Also, amino groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. A pharmaceutically acceptable salt may suitably be a salt chosen, e.g., among acid addition salts and basic salts. Examples of acid addition salts include chloride salts, citrate salts and acetate salts. Examples of basic salts include salts where the cation is selected among alkali metal cations, such as sodium or potassium ions, alkaline earth metal cations, such as calcium or magnesium ions, as well as substituted ammonium ions, such as ions of the type N(R1)(R2)(R3)(R4)+, where R1, R2, R3 and R4 independently will typically designate hydrogen, optionally substituted C1-6-alkyl or optionally substituted C2-6-alkenyl. Examples of relevant C1-6-alkyl groups include methyl, ethyl, 1-propyl and 2-propyl groups. Examples of C2-6-alkenyl groups of possible relevance include ethenyl, 1-propenyl and 2-propenyl. Other examples of pharmaceutically acceptable salts are described in “Remington's Pharmaceutical Sciences”, 17th edition, Alfonso R. Gennaro (Ed.), Mark Publishing Company, Easton, PA, USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66: 2 (1977). Also, for a review on suitable salts, see Handbook of Pharmaceutical Salts: Properties, Selection, and Use by Stahl and Wermuth (Wiley-VCH, 2002). Other suitable base salts are formed from bases which form non-toxic salts. Representative examples include the aluminum, arginine, benzathine, calcium, choline, diethylamine, diolamine, glycine, lysine, magnesium, meglumine, olamine, potassium, sodium, tromethamine, and zinc salts. Hemisalts of acids and bases may also be formed, e.g., hemisulphate and hemicalcium salts.

The term “alkyl” includes a straight chain or branched, noncyclic or cyclic, saturated aliphatic hydrocarbon containing from 1 to 24 carbon atoms. Representative saturated straight chain alkyls include, but are not limited to, methyl, ethyl, n-propyl, n-butyl, n-pentyl, n-hexyl, and the like, while saturated branched alkyls include, without limitation, isopropyl, sec-butyl, isobutyl, tert-butyl, isopentyl, and the like. Representative saturated cyclic alkyls include, but are not limited to, cyclopropyl, cyclobutyl, cyclopentyl, cyclohexyl, and the like, while unsaturated cyclic alkyls include, without limitation, cyclopentenyl, cyclohexenyl, and the like.

The term “thio”, “mercapto” or “sulfanyl” means an —SH group.

As used herein, a “therapeutically effective amount” of the peptide agonists of the invention is meant to describe a sufficient amount of the peptide agonist to treat an hepcidin-related disease, including but not limited to any of the diseases and disorders described herein (for example, a disease of iron metabolism). In particular embodiments, the therapeutically effective amount will achieve a desired benefit/risk ratio applicable to any medical treatment.

### Methods of Treatment Using Hepcidin Mimetics

Sickle cell disease (SCD) is an autosomal recessive disorder that affects a significant proportion (approximately 1 in 500 individuals). An A to T transversion in the 6th codon of the human β-globin gene changes a polar glutamic acid residue to a nonpolar valine in the 0-globin chain on the surface of HbS (α2β^{s}2) tetramers. The interaction of tetramers results in the formation of HbS polymers/fibers that cause RBCs to become rigid and nondeformable and to occlude small capillaries. Vaso-occlusive events cause severe tissue damage that can result in strokes, splenic infarction, kidney failure, liver and lung disorders, painful crises, and other complications. Erythrocyte sickling causes cells to become fragile, and lysis produces chronic anemia. Treatments available to manage symptoms include pain relievers for pain management; hydroxyurea (increases size of RBCs to prevent sickling); blood transfusions (may cause transfusional iron overload); bone marrow/stem cell transplants; and experimental therapies.

Hepcidin targets the major iron transporter ferroportin and causes its internalization and subsequent degradation. Hepcidin regulation is crucial for providing adequate iron needed for cellular functions while also preventing iron toxicity.

The present disclosure provides methods of treating SCD in a subject by administering a hepcidin mimetic or a pharmaceutically acceptable salt thereof or a composition containing a hepcidin mimetic or a pharmaceutically acceptable salt thereof and a pharmaceutically acceptable excipient or carrier. In certain embodiments, treatments with hepcidin mimetics are beneficial in improving disease-related complete blood count (CBC) parameters such as total white blood cell (WBC), total red blood cell (RBC), hemoglobin (HGB), hematocrit (HCT), mean corpuscular volume (MCV), mean cell hemoglobin (MCH), MCH concentration (MCHC), and etc. and serum biomarkers along with controlling tissue damage in SCD.

Different forms of SCD treatable with a hepcidin mimetic as described or disclosed herein or a pharmaceutically salt thereof, or a composition as described herein include, but are not limited to, the homozygous sickle-cell anemia (HbSS), the heterozygous sickle-cell trait (HbAC), and the compound heterozygous sickle-hemoglobin C, sickle-hemoglobin D (HbSD), sickle-hemoglobin E (HbSE), sickle-hemoglobin O (HbSO), sickle-beta-plus-thalassemia (HbSβ+thalassemia), sickle-beta-zero-thalassemia) (HbSβ₀ thalassemia).

In some embodiments, the disclosure provides a method for treating subtypes or genotypes of sickle cell disease selected from sickle cell anemia (HbSS), HbSβ0 thalassemia, HbSβ+thalassemia or hemoglobin SC disease (HbSC). The method comprises administering to the subject in need thereof an effective amount of hepcidin mimetic as disclosed or described herein or a pharmaceutically acceptable salt thereof or a composition containing a hepcidin mimetic or a pharmaceutically acceptable salt thereof or a composition comprising a hepcidin mimetic or a pharmaceutically acceptable salt thereof and a pharmaceutically acceptable excipient or carrier. In certain embodiments, the disease treatable with the methods described herein is HbSC,

In some embodiments, the disclosure provides a method for treating sickle cell anemia (SS), sickle hemoglobin-C disease (SC), sickle beta-plus thalassemia or sickle beta-zero Thalassemia in a subject. The method comprises administering to the subject in need thereof an effective amount of hepcidin mimetic as disclosed or described herein or a pharmaceutically acceptable salt thereof or a composition containing a hepcidin mimetic or a pharmaceutically acceptable salt thereof and a pharmaceutically acceptable excipient or carrier.

In some embodiments, the present disclosure provides a method of treating SCD in a subject, which comprises administering to the subject a hepcidin mimetic which is a peptide comprising or consisting of Formula (I):

R1-X—Y—R2 (I)
or a pharmaceutically acceptable salt or solvate thereof,
wherein
R1 is hydrogen, a C1-C6 alkyl, a C6-C12 aryl, a C1-C20 alkanoyl, or pGlu;
R2 is NH₂ or OH;
X is an amino acid sequence of Formula II:

X1-X2-X3-X4-X5-X6-X7-X8-X9-X10 (II)
wherein
X1 is Asp, Ala, Ida, pGlu, bhAsp, Leu, D-Asp, or absent;
X2 is Thr, Ala, or D-Thr;
X3 is His, Lys, D-His, or Lys;
X4 is Phe, Ala, Dpa, or D-Phe;
X5 is Pro, Gly, Arg, Lys, Ala, D-Pro, or bhPro;
X6 is Ile, Cys, Arg, Lys, D-Ile, or D-Cys;
X7 is Cys, Ile, Leu, Val, Phe, D-Ile, or D-Cys;
X8 is Ile, Arg, Phe, Gln, Lys, Glu, Val, Leu, or D-Ile;
X9 is Phe or bhPhe; and
X10 is Lys, Phe, or absent;
wherein if Y is absent, X7 is Ile; and
Y is an amino acid sequence of Formula III:

Y1-Y2-Y3-Y4-Y5-Y6-Y7-Y8-Y9-Y10-Y11-Y12-Y13-Y14-Y15 (III)
wherein
Y1 is Gly, Cys, Ala, Phe, Pro, Glu, Lys, D-Pro, Val, Ser, or absent;
Y2 is Pro, Ala, Cys, Gly, or absent;
Y3 is Arg, Lys, Pro, Gly, His, Ala, Trp, or absent;
Y4 is Ser, Arg, Gly, Trp, Ala, His, Tyr, or absent;
Y5 is Lys, Met, Arg, Ala, or absent;
Y6 is Gly, Ser, Lys, Ile, Ala, Pro, Val, or absent;
Y7 is Trp, Lys, Gly, Ala, Ile, Val, or absent;
Y8 is Val, Thr, Gly, Cys, Met, Tyr, Ala, Glu, Lys, Asp, Arg, or absent;
Y9 is Cys, Tyr, or absent;
Y10 is Met, Lys, Arg, Tyr, or absent;
Y11 is Arg, Met, Cys, Lys, or absent;
Y12 is Arg, Lys, Ala or absent;
Y13 is Arg, Cys, Lys, Val or absent;
Y14 is Arg, Lys, Pro, Cys, Thr or absent; and
Y15 is Thr, Arg or absent;
wherein the peptide comprising Formula I is optionally PEGylated on R1, X, or Y;
wherein a side chain of an amino acid of the peptide is optionally conjugated to a lipophilic substituent or polymeric moiety; and
wherein Ida is iminodiacetic acid, pGlu is pyroglutamic acid, bhAsp is β-homoaspartic acid, and bhPro is β-homoproline. In certain embodiments, the SCD is sickle cell anemia (SS), sickle hemoglobin-C disease (SC), sickle beta-plus thalassemia or sickle beta-zero Thalassemia.

In certain embodiments of the methods described herein, the peptide of formula (I) comprises or consists of a disulfide bond formed between two Cys amino acid residues present in the peptide, e.g., wherein the thiol groups on the side chain of two cysteine residues in the peptide form a disulfide bond. In one embodiment, the peptide has a disulfide bond formed between two Cys amino acid residues present in the peptide.

In certain embodiments of the methods described herein, R1 is hydrogen, isovaleric acid, isobutyric acid or acetyl.

In certain embodiments of compounds of formula (I), X is a peptide comprising or consisting of an amino acid sequence of Formula IV:

X1-Thr-His-X4-X5-X6-X7-X8-Phe-X10 (IV)
wherein
X1 is Asp, Ida, pGlu, bhAsp or absent;
X4 is Phe or Dpa;
X5 is Pro or bhPro;
X6 is Ile, Cys, or Arg;
X7 is Cys, Ile, Leu, or Val;
X8 is Ile, Lys, Glu, Phe, Gln, or Arg; and
X10 is Lys or absent.
   In certain embodiments, X is a peptide having an amino acid sequence of Formula IV.

In some embodiments of compounds of formula (I), X is a peptide comprising or consisting of an amino acid sequence of Formula V:

X1-Thr-His-X4-X5-Cys-Ile-X8-Phe-X10 (V)
wherein
X1 is Asp, Ida, pGlu, bhAsp, or absent;
X4 is Phe or Dpa;
X5 is Pro or bhPro;
X8 is Ile, Lys, Glu, Phe, Gln or Arg; and
X10 is Lys or absent.
   In certain embodiments, X is a peptide having an amino acid sequence of Formula V.

In some embodiments of the methods described herein, the hepcidin mimetic is a peptide comprising or consisting of an amino acid sequence of Formula VI:

R¹—X—Y—R² (VI)
or a pharmaceutically acceptable salt thereof,
wherein:
R¹ is hydrogen, isovaleric acid, isobutyric acid, or acetyl;
R² is NH₂ or OH;
X is an amino acid sequence of Formula VII:
X1-Thr-His-X4-X5-Cys-Ile-X8-Phe-X10 (VII)
wherein
X1 is Asp, Ida, pGlu, bhAsp, or absent;
X4 is Phe or Dpa;
X5 is Pro or bhPro;
X8 is Ile, Lys, Glu, Phe, Gln, or Arg; and
X10 is Lys or absent;
wherein Y is an amino acid sequence of Formula VIII:

Y1-Pro-Y3-Ser-Y5-Y6-Y7-Y8-Cys-Y10 (VIII)
wherein
Y1 is Gly, Glu, Val, or Lys;
Y3 is Arg or Lys;
Y5 is Arg or Lys;
Y6 is Gly, Ser, Lys, Ile, or Arg;
Y7 is Trp or absent;
Y8 is Val, Thr, Asp, Glu, or absent; and
Y10 is Lys or absent;
wherein the peptide comprises or consists of a disulfide bond between the two Cys;
wherein the peptide is optionally PEGylated on R¹, X, or Y;
wherein a side chain of an amino acid of the peptide is optionally conjugated to a lipophilic substituent or polymeric moiety; and
wherein Ida is iminodiacetic acid; pGlu is pyroglutamic acid; bhAsp is β-homoaspartic acid; and bhPro is β-homoproline.
   In certain embodiments, the peptide has formula VI, wherein the two Cys residues in the peptide form a disulfide bond.
   In other embodiments, the peptide has formula (VI), wherein the peptide is not cyclized.

In certain embodiments of the methods described herein, the hepcidin mimetic is a peptide comprising or consisting of one of the following amino acid sequences:

(SEQ ID NO: 47)
DTHFPICIFGPRSKGWVC;
(SEQ ID NO: 48)
DTHFPCIIFGPRSKGWVCK;
(SEQ ID NO: 49)
DTHFPCIIFEPRSKGWVCK;
(SEQ ID NO: 50)
DTHFPCIIFGPRSKGWACK;
(SEQ ID NO: 51)
DTHFPCIIFGPRSKGWVCKK;
(SEQ ID NO: 52)
DTHFPCIIFVCHRPKGCYRRVCR;
(SEQ ID NO: 53)
DTHFPCIKFGPRSKGWVCK;
(SEQ ID NO: 54)
DTHFPCIKFKPRSKGWVCK;
(SEQ ID NO: 55)
DTHFPCIIFGPRSRGWVCK;
(SEQ ID NO: 56)
DTHFPCIKFGPKSKGWVCK;
(SEQ ID NO: 57)
DTHFPCIKFEPRSKGCK;
(SEQ ID NO: 58)
DTHFPCIKFEPKSKGWECK;
(SEQ ID NO: 59)
DTHFPCIKFEPRSKKCK;
(SEQ ID NO: 60)
DTHFPCIKFEPRSKGCKK;
(SEQ ID NO: 61)
DTHFPCIKFKPRSKGCK;
(SEQ ID NO: 62)
DTHFPCIKFEPKSKGCK;
(SEQ ID NO: 63)
DTHFPCIKF;
(SEQ ID NO: 64)
DTHFPCIIF;
or
(SEQ ID NO: 65)
DTKFPCIIF,

wherein said peptide is optionally PEGylated on R1, X, or Y;
wherein a side chain of an amino acid of the peptide is optionally conjugated to a lipophilic substituent or polymeric moiety; and
wherein the peptide optionally comprises a disulfide bond between two Cys amino acid residues of the peptide. In certain embodiments, the peptide is cyclized through a disulfide bond formed between two Cys amino acid residues of the same peptide. In other embodiments, the peptide is linear, i.e., not cyclized through a disulfide bond.

In certain embodiments of the methods described herein, the peptide comprises or consists of one of the following sequences:

(SEQ ID NO: 1)
Isovaleric acid-DTHFPICIFGPRSKGWVC-NH₂;
(SEQ ID NO: 2)
Isovaleric acid-DTHFPCIIFGPRSKGWVCK-NH₂;
(SEQ ID NO: 3)
Isovaleric acid-DTHFPCIIFEPRSKGWVCK-NH₂;
(SEQ ID NO: 4)
Isovaleric acid-DTHFPCIIFGPRSKGWACK-NH₂;
(SEQ ID NO: 5)
Isovaleric acid-DTHFPCIIFGPRSKGWVCKK-NH₂;
(SEQ ID NO: 6)
Isovaleric acid-DTHFPCIIFVCHRPKGCYRRVCR-NH₂;
(SEQ ID NO: 7)
Isovaleric acid-DTHFPCI(K(PEG8))FGPRSKGWVCK-NH₂;
(SEQ ID NO: 8)
Isovaleric acid-DTHFPCIKF(K(PEG8))PRSKGWVCK-NH₂;
(SEQ ID NO: 9)
Isovaleric acid-DTHFPICIFGPRS(K(PEG8))GWVC-NH₂;
(SEQ ID NO: 10)
Isovaleric acid-DTHFPICIFGPRS(K(PEG4))GWVC-NH₂;
(SEQ ID NO: 11)
Isovaleric acid-DTHFPCIIFGPRSRGWVC(K(PEG8))-NH₂;
(SEQ ID NO: 12)
Isovaleric acid-DTHFPCIIFGPRSRGWVC(K(PEG4))-NH₂;
(SEQ ID NO: 13)
Isovaleric acid-DTHFPCIIFGPRSRGWVC(K(PEG2))-NH₂;
(SEQ ID NO: 14)
Isovaleric acid-DTHFPCI(K(Palm))FGPRSKGWVCK-NH₂;
(SEQ ID NO: 15)
Isovaleric acid-DTHFPCIKF)K(Palm))PRSKGWVCK-NH₂;
(SEQ ID NO: 16)
Isovaleric acid-DTHFPCIKFGP(K(Palm))SKGWVCK-NH₂;
(SEQ ID NO: 17)
Isovaleric acid-DTHFPCIKFGPRS(K(Palm))GWVCK-NH₂;
(SEQ ID NO: 18)
Isovaleric acid-DTHFPCIKFGPRSKGWVC(K(Palm))NH₂;
(SEQ ID NO: 19)
Isovaleric acid-DTHFPCI(K(PEG3-Palm))FGPRSKGWVCK-NH₂;
(SEQ ID NO: 20)
Isovaleric acid-DTHFPCIKF(K(PEG3-Palm))PRSKGWVCK-NH₂;
(SEQ ID NO: 21)
Isovaleric acid-DTHFPCIKFGP(K(PEG3-Palm))SKGWVCK-NH₂;
(SEQ ID NO: 22)
Isovaleric acid-DTHFPCIKFGPRS(K(PEG3-Palm))GWVCK-NH₂;
(SEQ ID NO: 23)
Isovaleric acid-DTHFPCIKFGPRSKGWVC(K(PEG3-Palm))-NH₂;
(SEQ ID NO: 24)
Isovaleric acid-DTHFPCIKFGPRSKGWVC(K(PEG8))-NH₂;
(SEQ ID NO: 25)
Isovaleric acid-DTHFPCI(K(isoGlu-Palm))FEPRSKGCK-NH₂;
(SEQ ID NO: 26)
Isovaleric acid-DTHFPCIKF-K(isoGlu-Palm)-PRSKGCK-NH₂;
(SEQ ID NO: 27)
Isovaleric acid-DTHFPCIKFEP(K(isoGlu-Palm))SKGCK-NH₂;
(SEQ ID NO: 28)
Isovaleric acid-DTHFPCIKFEP(K(isoGlu-Palm))SKGWECK-NH₂;
(SEQ ID NO: 29)
Isovaleric acid-DTHFPCIKFEPRS(K(isoGlu-Palm))GCK-NH₂;
(SEQ ID NO: 30)
Isovaleric acid-DTHFPCIKFEPRSK(K(isoGlu-Palm))CK-NH₂;
(SEQ ID NO: 31)
Isovaleric acid-DTHFPCIKFEPRSKGCK(K(isoGlu-Palm))-NH₂;
(SEQ ID NO: 32)
Isovaleric acid-DTHFPCI-K(Dapa-Palm)-FEPRSKGCK-NH₂;
(SEQ ID NO: 33)
Isovaleric acid-DTHFPCIK(F(Dapa-Palm))PRSKGCK-NH₂;
(SEQ ID NO: 34)
Isovaleric acid-DTHFPCIKFEP(K(Dapa-Palm))SKGCK-NH₂;
(SEQ ID NO: 35)
Isovaleric acid-DTHFPCIKFEPRS(K(Dapa-Palm))GCK-NH₂;
(SEQ ID NO: 36)
Isovaleric acid-DTHFPCIKFEPRSK(K(Dapa-Palm))CK-NH₂;
(SEQ ID NO: 37)
Isovaleric acid-DTHFPCIKFEPRSKGC(K(Dapa-Palm))K-NH₂;
(SEQ ID NO: 38)
Isovaleric acid-DTHFPCIKFEPRSKGC(K(Dapa-Palm))-NH₂;
(SEQ ID NO: 39)
Isovaleric acid-DTHFPCIKF(K(PEG11-Palm))PRSK[Sar]CK-NH₂;
(SEQ ID NO: 46)
Isovaleric_Acid-ETHFPCI(K(IsoGlu_Palm))FEPRSKGCK-NH₂;
(SEQ ID NO: 40)
Isolvaleric acid-DTHFPCIKF-NH₂;
(SEQ ID NO: 41)
Hy-DTHFPCIKF-NH₂;
(SEQ ID NO: 42)
Isolvaleric acid-DTHFPCIIF-NH₂;
(SEQ ID NO: 43)
Hy-DTHFPCIIKF-NH₂;
(SEQ ID NO: 44)
Isovaleric acid-DTKFPCIIF-NH₂;
or
(SEQ ID NO: 45)
Hy-DTKFPCIIF-NH₂,

optionally wherein the peptide comprises or consists of a disulfide bond between two Cys amino acid residues of the peptide. In certain embodiments, the peptide has a disulfide bond formed between two Cys amino acid residues of the peptide. In certain embodiments, the peptide is cyclized through a disulfide bond formed between two Cys amino acid residues of the same peptide. In other embodiments, the peptide is linear, i.e., not cyclized through a disulfide bond.

In certain embodiments of the methods described herein, the peptide has a structure selected from:

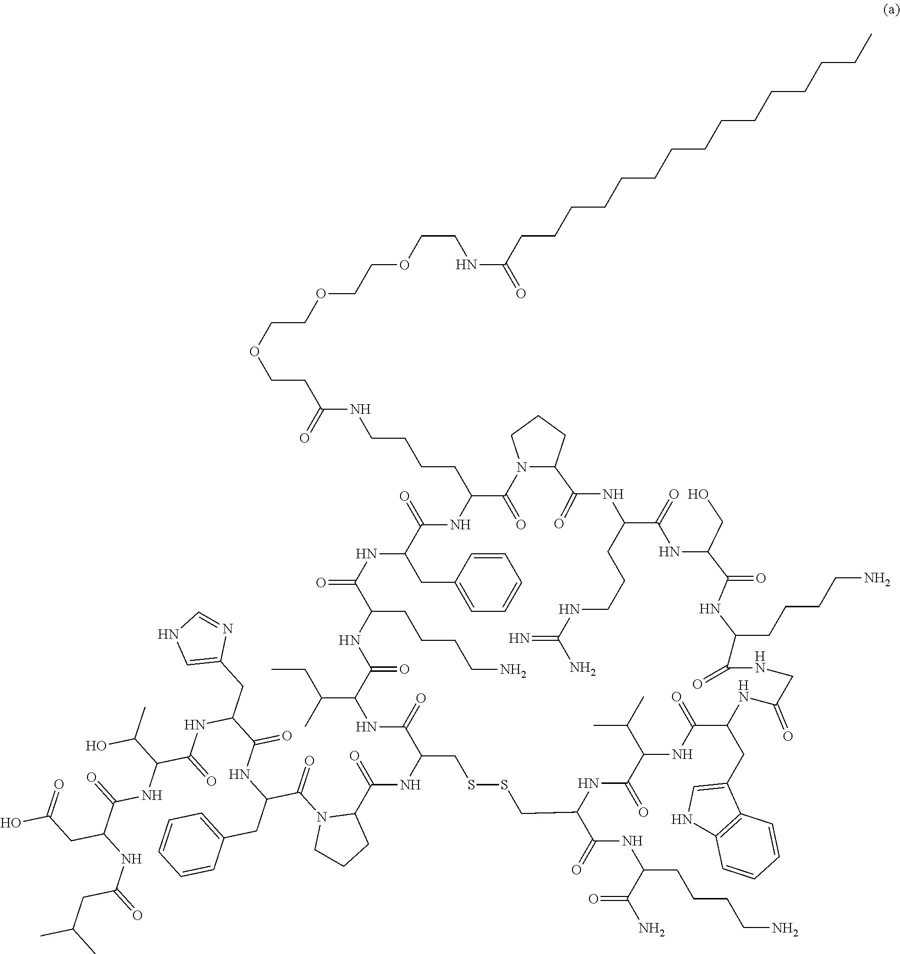 [SMILES: CCCCCCCCCCCCCCCC(=O)NCCOCCOCCOCCC(=O)NCCCCC1NC(=O)C(Cc2ccccc2)NC(=O)C(CCCCN)NC(=O)C(C(C)CC)NC(=O)C(NC(=O)C2CCCN2C(=O)C(Cc2ccccc2)NC(=O)C(Cc2c[nH]cn2)NC(=O)C(NC(=O)C(CC(=O)O)NC(=O)CC(C)C)C(C)O)CSSCC(C(=O)NC(CCCCN)C(N)=O)NC(=O)C(C(C)C)NC(=O)C(Cc2c[nH]c3ccccc23)NC(=O)CNC(=O)C(CCCCN)NC(=O)C(CO)NC(=O)C(CCCNC(=N)N)NC(=O)C2CCCN2C1=O]

Isovaleric acid-DTHFPCIKF(K(PEG3-Palm))PRSKGWVCK-NH₂ (compound 20; SEQ ID NO: 20)

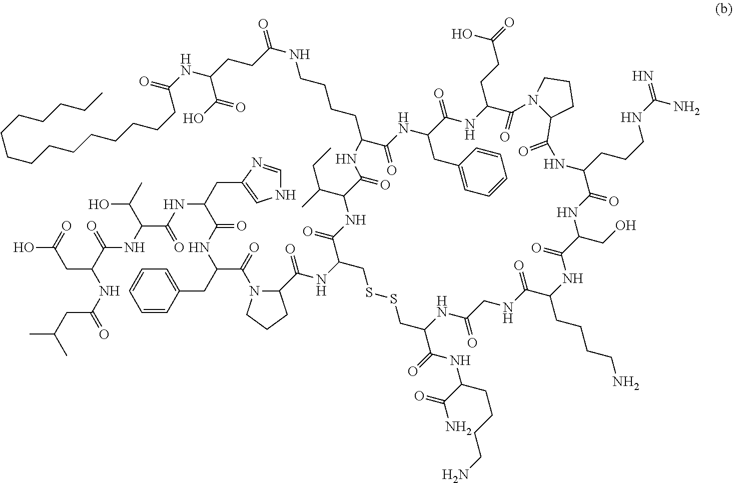 [SMILES: CCCCCCCCCCCCCCCC(=O)NC(CCC(=O)NCCCCC1NC(=O)C(C(C)CC)NC(=O)C(NC(=O)C2CCCN2C(=O)C(Cc2ccccc2)NC(=O)C(Cc2c[nH]cn2)NC(=O)C(NC(=O)C(CC(=O)O)NC(=O)CC(C)C)C(C)O)CSSCC(C(=O)NC(CCCCN)C(N)=O)NC(=O)CNC(=O)C(CCCCN)NC(=O)C(CO)NC(=O)C(CCCNC(=N)N)NC(=O)C2CCCN2C(=O)C(CCC(=O)O)NC(=O)C(Cc2ccccc2)NC1=O)C(=O)O]

Isovaleric acid-DTHFPCI(K(isoGlu-Palm))FEPRSKGCK-NH₂ (compound 25; SEQ ID NO: 25)

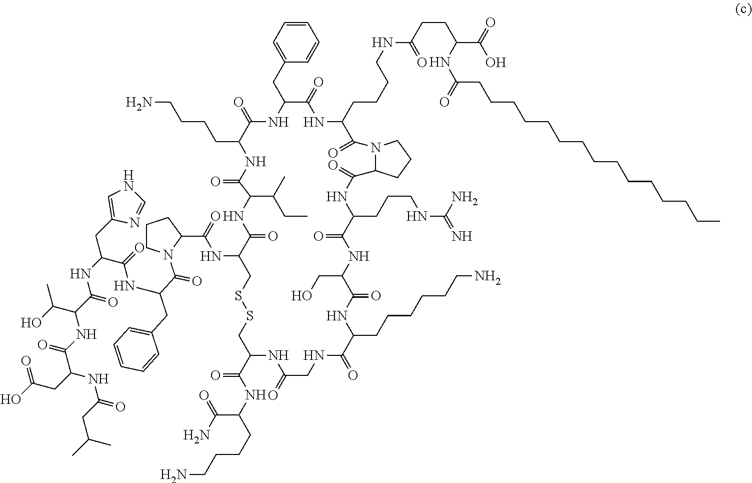 [SMILES: CCCCCCCCCCCCCCCC(=O)NC(CCC(=O)NCCCCC1NC(=O)C(Cc2ccccc2)NC(=O)C(CCCCN)NC(=O)C(C(C)CC)NC(=O)C(NC(=O)C2CCCN2C(=O)C(Cc2ccccc2)NC(=O)C(Cc2c[nH]cn2)NC(=O)C(NC(=O)C(CC(=O)O)NC(=O)CC(C)C)C(C)O)CSSCC(C(=O)NC(CCCCN)C(N)=O)NC(=O)CNC(=O)C(CCCCCCN)NC(=O)C(CO)NC(=O)C(CCCNC(=N)N)NC(=O)C2CCCN2C1=O)C(=O)O]

Isovaleric acid-DTHFPCIKF(K(isoGlu-Palm))PRSKGCK-NH₂ (compound 26; SEQ ID NO: 26)

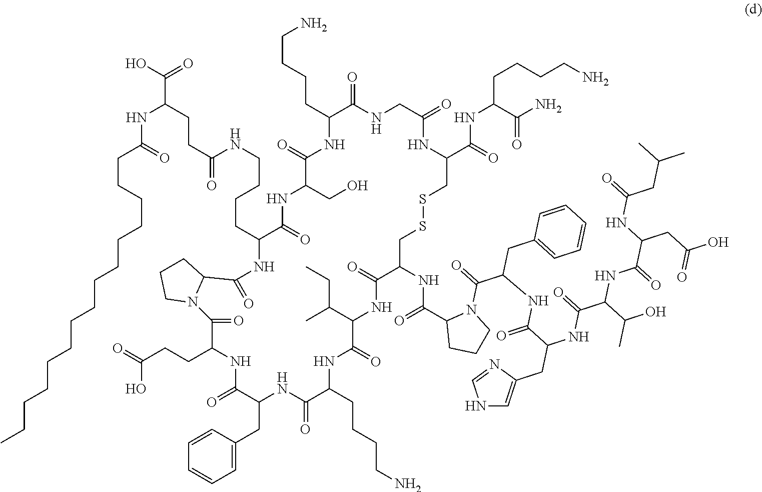 [SMILES: CCCCCCCCCCCCCCCC(=O)NC(CCC(=O)NCCCCC1NC(=O)C2CCCN2C(=O)C(CCC(=O)O)NC(=O)C(Cc2ccccc2)NC(=O)C(CCCCN)NC(=O)C(C(C)CC)NC(=O)C(NC(=O)C2CCCN2C(=O)C(Cc2ccccc2)NC(=O)C(Cc2c[nH]cn2)NC(=O)C(NC(=O)C(CC(=O)O)NC(=O)CC(C)C)C(C)O)CSSCC(C(=O)NC(CCCCN)C(N)=O)NC(=O)CNC(=O)C(CCCCN)NC(=O)C(CO)NC1=O)C(=O)O]

Isovaleric acid-DTHFPCIKFEP(K(isoGlu-Palm))SKGCK-NH₂ (compound 27; SEQ ID NO: 27)

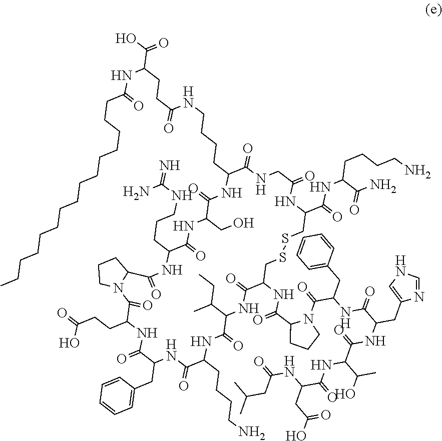 [SMILES: CCCCCCCCCCCCCCCC(=O)NC(CCC(=O)NCCCCC1NC(=O)C(CO)NC(=O)C(CCCNC(=N)N)NC(=O)C2CCCN2C(=O)C(CCC(=O)O)NC(=O)C(Cc2ccccc2)NC(=O)C(CCCCN)NC(=O)C(C(C)CC)NC(=O)C(NC(=O)C2CCCN2C(=O)C(Cc2ccccc2)NC(=O)C(Cc2c[nH]cn2)NC(=O)C(NC(=O)C(CC(=O)O)NC(=O)CC(C)C)C(C)O)CSSCC(C(=O)NC(CCCCN)C(N)=O)NC(=O)CNC1=O)C(=O)O]

Isovaleric acid-DTHFPCIKFEPRS(K(isoGlu-Palm))GCK-NH₂ (compound 28; SEQ ID NO: 28)

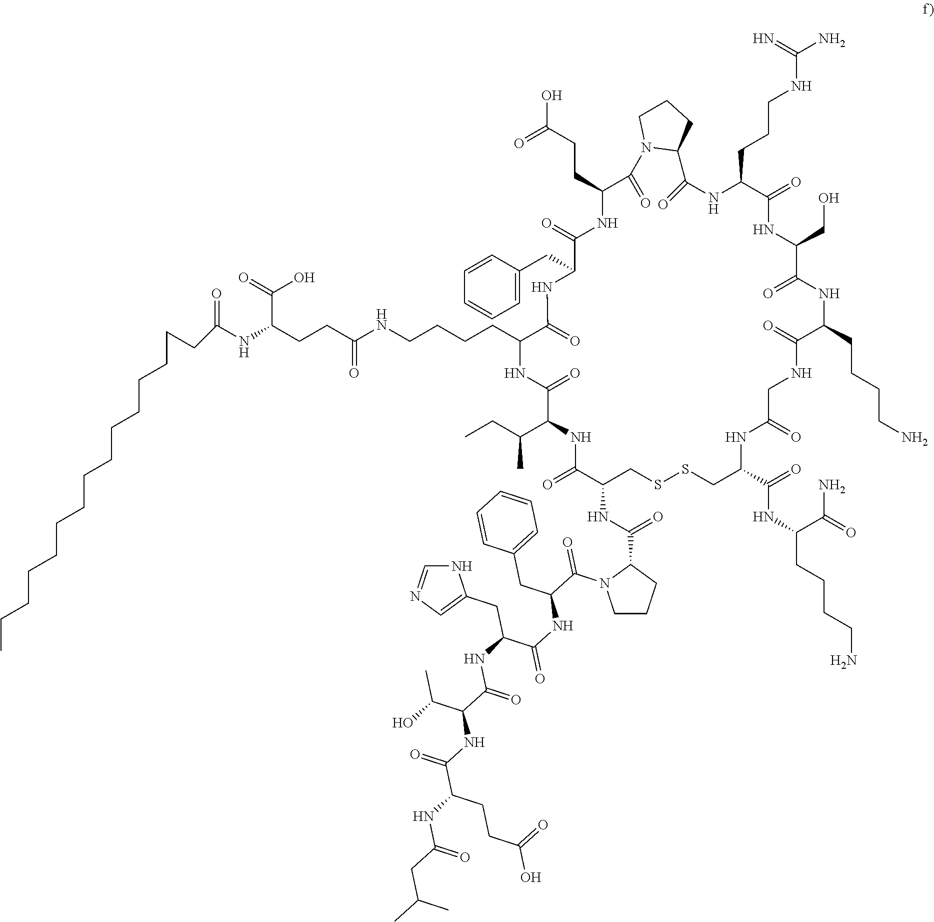 [SMILES: CCCCCCCCCCCCCCCC(=O)N[C@@H](CCC(=O)NCCCCC1NC(=O)[C@H]([C@@H](C)CC)NC(=O)[C@@H](NC(=O)[C@@H]2CCCN2C(=O)[C@H](Cc2ccccc2)NC(=O)[C@H](Cc2cnc[nH]2)NC(=O)[C@@H](NC(=O)[C@H](CCC(=O)O)NC(=O)CC(C)C)[C@@H](C)O)CSSC[C@@H](C(=O)N[C@@H](CCCCN)C(N)=O)NC(=O)CNC(=O)[C@H](CCCCN)NC(=O)[C@H](CO)NC(=O)[C@H](CCCNC(=N)N)NC(=O)[C@@H]2CCCN2C(=O)[C@H](CCC(=O)O)NC(=O)[C@H](Cc2ccccc2)NC1=O)C(=O)O]

Isovaleric_Acid-ETHFPCI(K(IsoGlu_Palm))FEPRSKGCK-NH₂ (compound 46; SEQ ID NO: 46)
wherein the amino acids are L-amino acids and wherein the thio groups on the side chains of two cysteine residues in each of compounds 20, 25, 26, 27, 28 and 46 taken together form a disulfide bond.

In another embodiment, the peptide is compound 2 or a pharmaceutically acceptable salt thereof.

In another embodiment, the peptide is compound 3 or a pharmaceutically acceptable salt thereof.

In another embodiment, the peptide is compound 7 or a pharmaceutically acceptable salt thereof.

In another embodiment, the peptide is compound 8 or a pharmaceutically acceptable salt thereof.

In another embodiment, the peptide is compound 11 or a pharmaceutically acceptable salt thereof.

In another embodiment, the peptide is compound 14 or a pharmaceutically acceptable salt thereof.

In another embodiment, the peptide is compound 15 or a pharmaceutically acceptable salt thereof.

In another embodiment, the peptide is compound 16 or a pharmaceutically acceptable salt thereof.

In another embodiment, the peptide is compound 18 or a pharmaceutically acceptable salt thereof.

In another embodiment, the peptide is compound 19 or a pharmaceutically acceptable salt thereof.

In another embodiment, the peptide is compound 20 or a pharmaceutically acceptable salt thereof.

In another embodiment, the peptide is compound 21 or a pharmaceutically acceptable salt thereof.

In another embodiment, the peptide is compound 22 or a pharmaceutically acceptable salt thereof.

In another embodiment, the peptide is compound 23 or a pharmaceutically acceptable salt thereof.

In another embodiment, the peptide is compound 24 or a pharmaceutically acceptable salt thereof.

In another embodiment, the peptide is compound 26 or a pharmaceutically acceptable salt thereof.

In another embodiment, the peptide is compound 27 or a pharmaceutically acceptable salt thereof.

In another embodiment, the peptide is compound 28 or a pharmaceutically acceptable salt thereof.

In another embodiment, the peptide is compound 32 or a pharmaceutically acceptable salt thereof.

In another embodiment, the peptide is compound 34 or a pharmaceutically acceptable salt thereof.

In some embodiments of the methods disclosed herein, the peptide is compound 25 or a pharmaceutically acceptable salt thereof.

In other embodiments of the methods disclosed herein, the peptide is compound 46 or a pharmaceutically acceptable salt thereof.

In some embodiments of the methods disclosed herein, the effective amount is from about 0.5 mg to about 100 mg, or about 0.5 mg to about 50 mg, or about 0.5 mg to about 35 mg, or about 1 mg to about 24 mg, e.g., about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.5 mg, about 2.0 mg, about 2.5 mg, about 3.0 mg, about 3.5 mg, about 4.0 mg, about 4.5 mg, about 5.0 mg, about 5.5 mg, about 6.0 mg, about 7.0 mg, about 7.5 mg, about 8.0 mg, about 9.0 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 40 mg, about 45 mg, about 50 mg, about 55 mg, about 60 mg, about 65 mg, about 70 mg, about 75 mg, about 80 mg, about 85 mg, about 90 mg, or about 100 mg of hepcidin compound as disclosed herein or a pharmaceutically acceptable salt thereof.

In some embodiments, the hepcidin mimetic as disclosed herein is administered to the subject about twice a week, about once a week, about once every other week, or about once a month. In particular embodiments, the subject is administered the hepcidin mimetic or pharmaceutically acceptable salt thereof about once every week. In particular embodiments, the hepcidin mimetic or a pharmaceutically acceptable salt thereof is administered to a subject about once every two weeks or about once a month. In some embodiments, the hepcidin mimetic or pharmaceutically acceptable salt thereof is administered multiple times over a period of time, e.g., a time period of at least six months, at least or about one year, at least or about two years, at least or about five years, or for the subject's lifetime.

In some embodiments of the methods disclosed herein, the hepcidin mimetic or pharmaceutically acceptable salt thereof or peptide is administered in a composition (e.g., a pharmaceutical composition), and in some embodiments, the hepcidin mimetic or pharmaceutically acceptable salt thereof or peptide (or composition) is administered via subcutaneous injection. In some embodiments, the hepcidin mimetic or pharmaceutically acceptable salt thereof or peptide (or composition) is administered about weekly over a period of time, e.g., as long as needed. In some embodiments, the hepcidin mimetic or peptide (or composition) is administered about every three days, about twice a week, about every four days, about every five days, about weekly, about once every two weeks, about once a month, about once every six weeks, about once every eight weeks, about once every two months, or about once every three months. In some embodiments, it is administered about once a week or about once every two weeks. In particular embodiments, it is administered about once a week. In some embodiments, it is administered about once every two weeks, about once a month, or about once every two months.

In some embodiments of the methods disclosed herein, treatment of SCD results in significant reduction of HCT in a dose dependent manner in the subject. In some embodiments, the subject's hematocrit level is ≤45%. In certain embodiments, the subject's hematocrit is maintained within a range of about 37.5% to about 45% (or within the acceptable range for the subject's sex and pregnancy status) over a period of time, e.g., for at least one month, at least two months, at least six months, or longer. In certain embodiments, the method or treatment regimen results in a decrease in hematocrit (HCT %) of at least 3%, at least 5%, at least 10% for at least one month, at least two months, at least three months, at least six months, or longer.

In some embodiments of the methods disclosed herein, treatment of SCD results in reduction of mean corpuscular volume (MCV) in the subject. In certain embodiments, the method of treating SCD results in a decrease in MCV of at least about 1 fL, about 3 fL, about 5 fL, about 10 fL, about 25 fL, about 20 fL, about 25 fL or about 30 fL. In some embodiments, the MCV decrease by at least 10%, at least 20%, at least 30%, at least 50%, at least 90%, or at least 95% during a treatment regimen, or for at least one month, at least two months, at least six months, or longer.

In some embodiments of the methods disclosed herein, treatment of SCD results in reduction of mean corpuscular hemoglobin (MCH) in the subject. In certain embodiments, the method of treating SCD results in a decrease in MCH of at least about 1, about 2 pg, about 3 pg, about 4 pg, about 5 pg, about 6 pg, about 7 pg, about 8pg, about 9 pg, or about 10 pg. In some embodiments, the MCH increases by at least 10%, at least 20%, at least 30%, at least 50%, at least 100%, or at least 200% during a treatment regimen, or for at least one month, at least two months, at least six months, or longer.

In some embodiments of the methods disclosed herein, treatment of SCD results in increase of mean corpuscular hemoglobin concentration (MCHC) in the subject. In certain embodiments, the method of treating SCD results in an increase in MCHC of at least about 1 g/d, about 2 g/d, about 3 g/d, about 4 g/d, about 5 g/d, about 6 g/d, about 7 g/d, about 8 g/d, about 9 g/d, about 10 g/d, about 11 g/d, about 12 g/d, about 13 g/d, about 14 g/d or about 15 g/d during a treatment regimen for at least one month, at least two months, at least three months, at least six months, or longer.

In some embodiments of the methods disclosed herein, treatment of SCD results in reduction of white blood cell (WBC) count in the subject. In certain embodiments, the method of treating SCD results in a decrease in WBC by at least about 1 K/uL, about 2 K/uL, about 3 K/uL, about 4 K/uL, about 5 K/uL, about 6 K/uL, about 7 K/uL, about 8 K/uL, about 9 K/uL, about 10 K/uL, about 15 K/uL, about 20 K/uL, about 25 K/uL, about 30 K/uL, or about 35 K/uL. In certain embodiments, the decrease in WCB is during a treatment regimen for at least one month, at least two months, at least three months, at least six months, or longer.

In some embodiments of the methods disclosed herein, treatment of SCD results in reduction of lymphocytes count in the subject. In certain embodiments, the method of treating SCD results in a decrease in lymphocyte count by at least about 1000/μL, about 2000/μL, about 3000/μL, about 5000/μL, about 10000, about 12000/μL, about 15000/μL, about 20000/μL, about 25000/μL, about 28000/μL, or about 30000/μL. In some embodiments, the lymphocyte count decreases by at least 5%, at least 10%, at least 20%, at least 30%, at least 50%, at least 90%, or at least 95% during a treatment regimen. In certain embodiments, the decrease in lymphocytes count is during a treatment regimen for at least one month, at least two months, at least three months, at least six months, or longer.

In some embodiments of the methods disclosed herein, treatment of SCD results in reduction of monocytes count in the subject. In certain embodiments, the method of treating SCD results in a decrease in monocytes count by at least about 100/μL, about 200/μL, about 300/μL, about 400/μL, about 500/μL, /μL 1000/μL, about 1200/μL, about 1500/μL, about 2000/μL, about 2500/μL, about 2800/μL, or about 3000/μL. In some embodiments, the monocyte count decreases by at least 5%, at least 10%, at least 20%, at least 30%, at least 50%, at least 90%, or at least 95% during a treatment regimen. In certain embodiments, the decrease in monocyte count is during a treatment regimen for at least one month, at least two months, at least three months, at least six months, or longer.

In some embodiments of the methods disclosed herein, treatment of SCD results in reduction of neutrophil count in the subject. In certain embodiments, the method of treating SCD results in a decrease in neutrophil count by at least about 200/μL, about 300/μL, about 400/μL, about 500/μL, about 1000/μL, about 2000/μL, about 2500/μL, about 2800/μL, about 3000/μL, about 4000/μL, about 5000/μL, about 6000/μL, about 7000/μL, about 7500/μL, about 8000/μL, about 8500/μL, about 9000/μL or 10000/μL. In some embodiments, the neutrophil count decreases by at least 5%, at least 10%, at least 20%, at least 30%, at least 50%, at least 70%, at least 80%, at least 90%, or at least 95% during a treatment regimen. In certain embodiments, the decrease in neutrophil count is during a treatment regimen for at least one month, at least two months, at least three months, at least six months, or longer.

In some embodiments of the methods disclosed herein, treatment of SCD results in reduction of serum lactate dehydrogenase (LDH) in the subject. In certain embodiments, the method of treating SCD results in a decrease in LDH by at least about 100, about 200/μL, about 300/μL, about 400/μL, about 500/μL, about 600/μL, about 700/μL, about 800/μL, about 900/μL, or about 1000/μL. In some embodiments, the LDH decreases by at least 5%, at least 10%, at least 20%, at least 30%, at least 50%, at least 70%, at least 80%, at least 90%, or at least 95% during a treatment regimen. In certain embodiments, the decrease in LDH is during a treatment regimen for at least one month, at least two months, at least three months, at least six months, or longer.

In some embodiments of the methods disclosed herein, treatment of SCD results in reduction of total bilirubin concentration in the subject. In certain embodiments, the method of treating SCD results in a decrease in total bilirubin concentration by at least about 0.5 mg/μL, about 1.0 mg/μL, about 1.3 mg/μL, about 1.5 mg/μL, about 2 mg/μL, about 2.5 mg/μL, about 3 mg/μL, about 3.5 mg/μL or about 4 mg/μL. In some embodiments, the total bilirubin concentration decreases by about at least 5%, at least 10%, at least 20%, at least 30%, at least 50%, at least 70%, at least 80%, at least 90%, or at least 95% during a treatment regimen. In certain embodiments, the decrease in bilirubin concentration is during a treatment regimen for at least one month, at least two months, at least three months, at least six months, or longer.

In some embodiments of the methods disclosed herein, treatment of SCD results in reduction of reticulocyte counts in the subject. In certain embodiments, the method of treating SCD results in a decrease in absolute reticulocyte counts by at least about 500 (K/uL), about 600 (K/uL), about 700 (K/uL), about 800 (K/uL), about 900 (K/uL), about 1000 (K/uL), about 1500 (K/uL), about 2000 (K/uL) or about 2500 (K/uL). In some embodiments, the reticulocyte decreases by at least about 5%, 10%, 15%, 20%, 25%, 30%, 35% or 40% during a treatment regimen. In certain embodiments, the decrease in reticulocyte is during a treatment regimen for at least one month, at least two months, at least three months, at least six months, or longer.

In some embodiments, treatment of SCD with a peptide as disclosed herein reduces MCHC, hemichrome aggregates and increased red blood cell lifespan.

In some embodiments, a treatment regimen comprises two or more, three or more, four or more, or serial administrations of a hepcidin mimetic over a period of time, e.g., about once a week or about once every two weeks for the period of time, for at least one month, at least two months, at least six months, or longer.

In some embodiments, the sickle cell diseases described herein can be treated by administering to a subject any of the hepcidin mimetics disclosed in any of U.S. Pat. Nos. 9,822,157, 10,030,061 and 9,315,545; PCT application publications, WO15200916, WO17117411, WO18048944, WO18128828, WO17068089, WO2017117411, WO2019157268, WO2022026629, WO2022026631 and WO2022026633, each of which is incorporated herein by reference in its entirety for all purposes.

### Administration and Pharmaceutical Composition

Administration of the peptide of Formula I, II, III, IV, V, VI, VII, or VIII, or a compound disclosed herein or recited in the claims, or compound 25 or compound 46, or a pharmaceutically acceptable salt thereof, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin dosages (which can be in capsules or tablets), powders, solutions, suspensions, or aerosols, or the like, specifically in unit dosage forms suitable for simple administration of precise dosages. In one embodiment, the peptide is administered subcutaneously.

The compositions will include a conventional pharmaceutical carrier or excipient and a compound of Formula I, II, III, IV, V, VI, VII, or VIII, or a compound disclosed herein or recited in the claims, or compound 25 or compound 46, or a pharmaceutically acceptable salt thereof as the/an active agent, and, in addition, may include a carrier, excipient, vehicle, and/or adjuvants.

Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the hepcidin mimetics or peptides disclosed herein may also contain minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, etc.

In some embodiments, the hepcidin mimetics or pharmaceutical compositions comprising a hepcidin mimetic as disclosed herein are in unit dosage form. In such forms, the composition is divided into unit doses containing appropriate quantities of the active component or components. The unit dosage form may be presented as a packaged preparation, the package containing discrete quantities of the preparation, for example, packaged tablets, capsules or powders in vials or ampoules. The unit dosage form may also be, e.g., a capsule, cachet or tablet in itself, or it may be an appropriate number of any of these packaged forms. A unit dosage form may also be provided in single-dose injectable form, for example in the form of a pen device containing a liquid-phase (typically aqueous) composition. Compositions may be formulated for any suitable route and means of administration, e.g., any one of the routes and means of administration disclosed herein.

In certain embodiments, the hepcidin mimetic, or the pharmaceutical composition comprising a hepcidin mimetic, is suspended in a sustained-release matrix. A sustained-release matrix, as used herein, is a matrix made of materials, usually polymers, which are degradable by enzymatic or acid-base hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid) polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. One embodiment of a biodegradable matrix is a matrix of one of either polylactide, polyglycolide, or polylactide co-glycolide (co-polymers of lactic acid and glycolic acid).

In certain embodiments, the compositions are administered parenterally, subcutaneously or orally. In some embodiments, the compositions are administered orally, intracisternally, intravaginally, intraperitoneally, intrarectally, topically (as by powders, ointments, drops, suppository, or transdermal patch, including delivery intravitreally, intranasally, and via inhalation) or buccally. The term “parenteral” as used herein refers to modes of administration which include intravenous, intramuscular, intraperitoneal, intrasternal, subcutaneous, intradermal and intra-articular injection and infusion. Accordingly, in certain embodiments, the compositions are formulated for delivery by any of these routes of administration.

In certain embodiments, pharmaceutical compositions for parenteral injection comprise pharmaceutically acceptable sterile aqueous or nonaqueous solutions, dispersions, suspensions or emulsions, or sterile powders, for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, beta-cyclodextrin, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants. These compositions may also contain adjuvants such as preservative, wetting agents, emulsifying agents, and dispersing agents. Prolonged absorption of an injectable pharmaceutical form may be brought about by the inclusion of agents which delay absorption, such as aluminum monostearate and gelatin.

Injectable depot forms include those made by forming microencapsule matrices of the hepcidin mimetic in one or more biodegradable polymers such as polylactide-polyglycolide, poly(orthoesters), poly(anhydrides), and (poly)glycols, such as PEG. Depending upon the ratio of peptide to polymer and the nature of the particular polymer employed, the rate of release of the hepcidin mimetic can be controlled. Depot injectable formulations are also prepared by entrapping the hepcidin mimetic in liposomes or microemulsions compatible with body tissues.

The injectable formulations may be sterilized, for example, by filtration through a bacterial-retaining filter, or by incorporating sterilizing agents in the form of sterile solid compositions which can be dissolved or dispersed in sterile water or other sterile injectable medium just prior to use.

Hepcidin mimetics and peptides as disclosed herein may also be administered in liposomes or other lipid-based carriers. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a hepcidin mimetic disclosed herein, stabilizers, preservatives, excipients, and the like. In certain embodiments, the lipids comprise phospholipids, including the phosphatidyl cholines (lecithins) and serines, both natural and synthetic. Methods to form liposomes are known in the art.

Pharmaceutical compositions to be used in the invention suitable for parenteral administration may comprise sterile aqueous solutions and/or suspensions of the peptide inhibitors made isotonic with the blood of the recipient, generally using sodium chloride, glycerin, glucose, mannitol, sorbitol, and the like.

In some embodiments, the pharmaceutical compositions and hepcidin mimetics as disclosed herein can be prepared for oral administration according to any of the methods, techniques, and/or delivery vehicles described herein. Further, one having skill in the art will appreciate that the hepcidin mimetics may be modified or integrated into a system or delivery vehicle that is not disclosed herein yet is well known in the art and compatible for use in oral delivery of peptides.

In certain embodiments, formulations for oral administration may comprise adjuvants (e.g. resorcinols and/or nonionic surfactants such as polyoxyethylene oleyl ether and n-hexadecylpolyethylene ether) to artificially increase the permeability of the intestinal walls, and/or enzymatic inhibitors (e.g. pancreatic trypsin inhibitors, diisopropylfluorophosphate (DFF) or trasylol) to inhibit enzymatic degradation. In certain embodiments, the hepcidin mimetic of a solid-type dosage form for oral administration can be mixed with at least one additive, such as sucrose, lactose, cellulose, mannitol, trehalose, raffinose, maltitol, dextran, starches, agar, alginates, chitins, chitosans, pectins, gum tragacanth, gum arabic, gelatin, collagen, casein, albumin, synthetic or semisynthetic polymer, or glyceride. These dosage forms can also contain other type(s) of additives, e.g., inactive diluting agent, lubricant such as magnesium stearate, paraben, preserving agent such as sorbic acid, ascorbic acid, alpha-tocopherol, antioxidants such as cysteine, disintegrators, binders, thickeners, buffering agents, pH adjusting agents, sweetening agents, flavoring agents or perfuming agents.

In some embodiments, oral dosage forms or unit doses compatible for use with the hepcidin mimetics as disclosed herein may include a mixture of hepcidin mimetics and nondrug components or excipients, as well as other non-reusable materials that may be considered either as an ingredient or packaging. Oral compositions may include at least one of a liquid, a solid, and a semi-solid dosage forms. In some embodiments, an oral dosage form is provided comprising an effective amount of a hepcidin mimetic, wherein the dosage form comprises at least one of a pill, a tablet, a capsule, a gel, a paste, a drink, a syrup, ointment, and suppository. In some instances, an oral dosage form is provided that is designed and configured to achieve delayed release of the hepcidin mimetic in the subject's small intestine and/or colon.

In one embodiment, an oral pharmaceutical composition comprising a hepcidin mimetic which comprises an enteric coating that is designed to delay release of the hepcidin mimetic in the small intestine. In at least some embodiments, a pharmaceutical composition is provided which comprises a hepcidin mimetic as disclosed herein and a protease inhibitor, such as aprotinin, in a delayed release pharmaceutical formulation. In some instances, pharmaceutical compositions of the instant invention comprise an enteric coat that is soluble in gastric juice at a pH of about 5.0 or higher. In at least one embodiment, a pharmaceutical composition is provided comprising an enteric coating comprising a polymer having dissociable carboxylic groups, such as derivatives of cellulose, including hydroxypropylmethyl cellulose phthalate, cellulose acetate phthalate and cellulose acetate trimellitate and similar derivatives of cellulose and other carbohydrate polymers.

In one embodiment, a pharmaceutical composition comprising a hepcidin mimetic as disclosed herein is provided in an enteric coating, the enteric coating being designed to protect and release the pharmaceutical composition in a controlled manner within the subject's lower gastrointestinal system, and to avoid systemic side effects. In addition to enteric coatings, the hepcidin mimetics disclosed herein can be encapsulated, coated, engaged or otherwise associated within any compatible oral drug delivery system or component. For example, in some embodiments a hepcidin mimetic as disclosed herein is provided in a lipid carrier system comprising at least one of polymeric hydrogels, nanoparticles, microspheres, micelles, and other lipid systems.

To overcome peptide degradation in the small intestine, some embodiments of the present invention comprise a hydrogel polymer carrier system in which a hepcidin mimetic as disclosed herein is contained, whereby the hydrogel polymer protects the hepcidin mimetic from proteolysis in the small intestine and/or colon. The hepcidin mimetics disclosed herein may further be formulated for compatible use with a carrier system that is designed to increase the dissolution kinetics and enhance intestinal absorption of the peptide. These methods include the use of liposomes, micelles and nanoparticles to increase GI tract permeation of peptides.

Various bioresponsive systems may also be combined with one or more hepcidin mimetic as disclosed herein to provide a pharmaceutical agent for oral delivery. In some embodiments, a hepcidin mimetic as disclosed herein is used in combination with a bioresponsive system, such as hydrogels and mucoadhesive polymers with hydrogen bonding groups (e.g., PEG, poly(methacrylic) acid [PMAA], cellulose, Eudragit®, chitosan and alginate) to provide a therapeutic agent for oral administration. Other embodiments include a method for optimizing or prolonging drug residence time for a hepcidin mimetic disclosed herein, wherein the surface of the hepcidin mimetic surface is modified to comprise mucoadhesive properties through hydrogen bonds, polymers with linked mucins or/and hydrophobic interactions. These modified peptide molecules may demonstrate increase drug residence time within the subject, in accordance with a desired feature of the invention. Moreover, targeted mucoadhesive systems may specifically bind to receptors at the enterocytes and M-cell surfaces, thereby further increasing the uptake of particles containing the hepcidin mimetic.

Other embodiments comprise a method for oral delivery of a hepcidin mimetic disclosed herein, wherein the hepcidin mimetic is provided to a subject in combination with permeation enhancers that promote the transport of the peptides across the intestinal mucosa by increasing paracellular or transcellular permeation. For example, in one embodiment, a permeation enhancer is combined with a hepcidin mimetic, wherein the permeation enhancer comprises at least one of a long-chain fatty acid, a bile salt, an amphiphilic surfactant, and a chelating agent. In one embodiment, a permeation enhancer comprising sodium N-[hydroxybenzoyl)amino] caprylate is used to form a weak noncovalent association with the hepcidin mimetic of the instant invention, wherein the permeation enhancer favors membrane transport and further dissociation once reaching the blood circulation. In another embodiment, a hepcidin mimetic disclosed herein is conjugated to oligoarginine, thereby increasing cellular penetration of the peptide into various cell types. Further, in one embodiment a noncovalent bond is formed between a peptide disclosed herein and a permeation enhancer selected from the group consisting of a cyclodextrin (CD) and a dendrimers, wherein the permeation enhancer reduces peptide aggregation and increasing stability and solubility for the hepcidin mimetic molecule.

In other embodiments, provided herewith is a method for treating a subject with a hepcidin mimetic disclosed herein having an increased half-life. In one embodiment, the present disclosure provides a hepcidin mimetic having a half-life of at least several hours to one day in vitro or in vivo (e.g., when administered to a human subject) sufficient for daily (q.d.) or twice daily (b.i.d.) dosing of a therapeutically effective amount. In another embodiment, the hepcidin mimetic has a half-life of three days or longer sufficient for weekly (q.w.) dosing of a therapeutically effective amount. Further, in another embodiment, the hepcidin mimetic has a half-life of eight days or longer sufficient for bi-weekly (b.i.w.) or monthly dosing of a therapeutically effective amount. In another embodiment, the hepcidin mimetic is derivatized or modified such that is has a longer half-life as compared to the underivatized or unmodified hepcidin mimetic. In another embodiment, the hepcidin mimetic contains one or more chemical modifications to increase serum half-life.

### Dosages

The hepcidin mimetics of this disclosure, or their pharmaceutically acceptable salts or solvates, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The hepcidin mimetics disclosed herein, can be administered to a subject at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

The total daily usage of the hepcidin mimetics and compositions disclosed herein can be decided by the attending physician within the scope of sound medical judgment. The specific therapeutically effective dose level for any particular subject will depend upon a variety of factors including: a) the disorder being treated and the severity of the disorder; b) activity of the specific compound employed; c) the specific composition employed, the age, body weight, general health, sex and diet of the patient; d) the time of administration, route of administration, and rate of excretion of the specific hepcidin mimetic employed; e) the duration of the treatment; f) drugs used in combination or coincidental with the specific hepcidin mimetic employed, and like factors well known in the medical arts.

In some embodiments, the total daily dose of the hepcidin mimetics disclosed herein to be administered to a human or other mammal host in single or divided doses may be in amounts, for example, from 0.0001 to 300 mg/kg body weight daily or 1 to 300 mg/kg body weight daily. In certain embodiments, a dosage of a hepcidin mimetic disclosed herein is in the range from about 0.0001 to about 100 mg/kg body weight per day, such as from about 0.0005 to about 50 mg/kg body weight per day, such as from about 0.001 to about 10 mg/kg body weight per day, e.g. from about 0.01 to about 1 mg/kg body weight per day, or from about 0.1 to about 10 mg/kg body weight per day, or from about 0.1 to about 35 mg/kg body weight per day, or from about 0.5 to about 25 mg/kg body weight per day administered in one or more doses, such as from one to three doses.

In some embodiments, a total dosage is about 10 mg to about 100 mg, or about 10 mg to about 70 mg, about 10 mg to about 60 mg, about 20 mg to about 50 mg, about 20 mg to about 40 mg, about 30 mg, about 25 mg, about 20 mg, about 15 mg, or about 10 mg, e.g., for a human patient. In some embodiments, the hepcidin mimetic is provided to the subject once a week. In another some embodiments, the hepcidin mimetic is provided to the subject twice a week e.g., for a human patient.

In a more particular embodiments, a total dosage is about 10 mg, about 15 mg, about 20 mg, about 25 mg, about 30 mg, about 40 mg, about 50 mg, about 60 mg, about 70 mg, or about 80 mg once or twice a week for a human patient. In a more particular embodiments, a total dosage is about 10 mg, about 15 mg, about 20 mg, about 25 mg, about 30 mg, about 40 mg, about 50 mg, about 60 mg, about 70 mg, or about 80 mg about every other week or about once a month for a human patient.

In various embodiments, a hepcidin mimetic as disclosed herein may be administered continuously (e.g. by intravenous administration or another continuous drug administration method), or may be administered to a subject at intervals, typically at regular time intervals, depending on the desired dosage and the pharmaceutical composition selected by the skilled practitioner for the particular subject. Regular administration dosing intervals include, e.g., once daily, twice daily, once every two, three, four, five or six days, once or twice weekly, once or twice monthly, and the like.

Such regular hepcidin mimetic administration regimens of the invention may, in certain circumstances such as, e.g., during chronic long-term administration, be advantageously interrupted for a period of time so that the medicated subject reduces the level of or stops taking the medication, often referred to as taking a “drug holiday.” Drug holidays are useful for, e.g., maintaining or regaining sensitivity to a drug especially during long-term chronic treatment, or to reduce unwanted side-effects of long-term chronic treatment of the subject with the drug. The timing of a drug holiday depends on the timing of the regular dosing regimen and the purpose for taking the drug holiday (e.g., to regain drug sensitivity and/or to reduce unwanted side effects of continuous, long-term administration). In some embodiments, the drug holiday may be a reduction in the dosage of the drug (e.g. to below the therapeutically effective amount for a certain interval of time). In other embodiments, administration of the drug is stopped for a certain interval of time before administration is started again using the same or a different dosing regimen (e.g. at a lower or higher dose and/or frequency of administration). A drug holiday of the invention may thus be selected from a wide range of time-periods and dosage regimens. An exemplary drug holiday is two or more days, one or more weeks, or one or more months, up to about 24 months of drug holiday. So, for example, a regular daily dosing regimen with a peptide, a peptide mimetic, or a dimer of the invention may, for example, be interrupted by a drug holiday of a week, or two weeks, or four weeks, after which time the preceding, regular dosage regimen (e.g. a daily or a weekly dosing regimen) is resumed. A variety of other drug holiday regimens are envisioned to be useful for administering the hepcidin mimetics of the invention.

Thus, the hepcidin mimetic may be delivered via an administration regime which comprises two or more administration phases separated by respective drug holiday phases.

During each administration phase, the hepcidin mimetic is administered to the recipient subject in a therapeutically effective amount according to a pre-determined administration pattern. The administration pattern may comprise continuous administration of the drug to the recipient subject over the duration of the administration phase. Alternatively, the administration pattern may comprise administration of a plurality of doses of the hepcidin mimetic to the recipient subject, wherein said doses are spaced by dosing intervals.

A dosing pattern may comprise at least two doses per administration phase, at least five doses per administration phase, at least 10 doses per administration phase, at least 20 doses per administration phase, at least 30 doses per administration phase, or more.

Said dosing intervals may be regular dosing intervals, which may be as set out above, including once daily, twice daily, once every two, three, four, five or six days, once or twice weekly, once or twice monthly, or a regular and even less frequent dosing interval, depending on the particular dosage formulation, bioavailability, and pharmacokinetic profile of the hepcidin mimetics as disclosed herein.

An administration phase may have a duration of at least two days, at least a week, at least 2 weeks, at least 4 weeks, at least a month, at least 2 months, at least 3 months, at least 6 months, or more.

Where an administration pattern comprises a plurality of doses, the duration of the following drug holiday phase is longer than the dosing interval used in that administration pattern. Where the dosing interval is irregular, the duration of the drug holiday phase may be greater than the mean interval between doses over the course of the administration phase. Alternatively the duration of the drug holiday may be longer than the longest interval between consecutive doses during the administration phase.

The duration of the drug holiday phase may be at least twice that of the relevant dosing interval (or mean thereof), at least 3 times, at least 4 times, at least 5 times, at least 10 times, or at least 20 times that of the relevant dosing interval or mean thereof.

Within these constraints, a drug holiday phase may have a duration of at least two days, at least a week, at least 2 weeks, at least 4 weeks, at least a month, at least 2 months, at least 3 months, at least 6 months, or more, depending on the administration pattern during the previous administration phase.

An administration regime comprises at least 2 administration phases. Consecutive administration phases are separated by respective drug holiday phases. Thus, the administration regime may comprise at least 3, at least 4, at least 5, at least 10, at least 15, at least 20, at least 25, or at least 30 administration phases, or more, each separated by respective drug holiday phases.

Consecutive administration phases may utilise the same administration pattern, although this may not always be desirable or necessary. However, if other drugs or active agents are administered in combination with a hepcidin mimetic disclosed herein, then typically the same combination of drugs or active agents is given in consecutive administration phases. In certain embodiments, the recipient subject is human.

In addition to the methods described in the Examples herein, the hepcidin mimetics of the present invention may be produced using methods known in the art including chemical synthesis, biosynthesis or in vitro synthesis using recombinant DNA methods, and solid phase synthesis. See e.g. Kelly & Winkler (1990) Genetic Engineering Principles and Methods, vol. 12, J. K. Setlow ed., Plenum Press, NY, pp. 1-19; Merrifield (1964) J Amer Chem Soc 85:2149; Houghten (1985) PNAS USA 82:5131-5135; and Stewart & Young (1984) Solid Phase Peptide Synthesis, 2ed. Pierce, Rockford, IL, which are herein incorporated by reference. The hepcidin mimetics of the present invention may be purified using protein purification techniques known in the art such as reverse phase high-performance liquid chromatography (HPLC), ion-exchange or immunoaffinity chromatography, filtration or size exclusion, or electrophoresis. See Olsnes, S. and A. Pihl (1973) Biochem. 12(16):3121-3126; and Scopes (1982) Protein Purification, Springer-Verlag, NY, which are herein incorporated by reference. Alternatively, the hepcidin mimetics of the present invention may be made by recombinant DNA techniques known in the art. Thus, polynucleotides that encode the polypeptides of the present invention are contemplated herein. In certain preferred embodiments, the polynucleotides are isolated. As used herein “isolated polynucleotides” refers to polynucleotides that are in an environment different from that in which the polynucleotide naturally occurs.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of Formula I, or a pharmaceutically acceptable salt thereof, for treatment of a disease-state in accordance with the teachings of this disclosure.

Peptides disclosed herein, including hepcidin mimetics, may be produced using methods known in the art including chemical synthesis, biosynthesis or in vitro synthesis using recombinant DNA methods, and solid phase synthesis. See e.g., PCT Application Publication Nos. WO 2014/145561 and WO 2015/200916; Kelly & Winkler (1990) Genetic Engineering Principles and Methods, vol. 12, J. K. Setlow ed., Plenum Press, NY, pp. 1-19; Merrifield (1964) J Amer Chem Soc 85:2149; Houghten (1985) PNAS USA 82:5131-5135; and Stewart & Young (1984) Solid Phase Peptide Synthesis, 2ed. Pierce, Rockford, IL, which are herein incorporated by reference. The peptides disclosed herein may be purified using protein purification techniques known in the art such as reverse phase high-performance liquid chromatography (HPLC), ion-exchange or immunoaffinity chromatography, filtration or size exclusion, or electrophoresis. See Olsnes, S. and A. Pihl (1973) Biochem. 12(16):3121-3126; and Scopes (1982) Protein Purification, Springer-Verlag, NY, which are herein incorporated by reference. Alternatively, the peptides may be made by recombinant DNA techniques known in the art.

In certain embodiments, peptides disclosed herein may be PEGylated. As used herein, “Polyethylene glycol” or “PEG” is a polyether compound of general Formula H—(O—CH2-CH2)n-OH. PEGs are also known as polyethylene oxides (PEOs) or polyoxyethylenes (POEs), depending on their molecular weight. PEG, PEO, or POE, as used herein, refers to an oligomer or polymer of ethylene oxide. The three names are chemically synonymous, but PEG has tended to refer to oligomers and polymers with a molecular mass below 20,000 Da, PEO to polymers with a molecular mass above 20,000 Da, and POE to a polymer of any molecular mass. PEG and PEO are liquids or low-melting solids, depending on their molecular weights. Throughout this disclosure, the three names are used indistinguishably. PEGs are prepared by polymerization of ethylene oxide and are commercially available over a wide range of molecular weights from 300 Da to 10,000,000 Da. While PEG and PEO with different molecular weights find use in different applications, and have different physical properties (e.g., viscosity) due to chain length effects, their chemical properties are nearly identical. PEG moieties include polyethylene glycols (PEG), homo- or co-polymers of PEG, a monomethyl-substituted polymer of PEG (mPEG), or polyoxyethylene glycerol (POG). See, for example, Int. J. Hematology 68:1 (1998); Bioconjugate Chem. 6:150 (1995); and Crit. Rev. Therap. Drug Carrier Sys. 9:249 (1992). Also encompassed are PEGs that are prepared for purpose of half life extension, for example, mono-activated, alkoxy-terminated polyalkylene oxides (POAs) such as mono-methoxy-terminated polyethyelene glycols (mPEGs); bis activated polyethylene oxides (glycols) or other PEG derivatives are also contemplated. Suitable PEGs will vary substantially by weights, e.g., ranging from about 200 Da to about 40,000 Da or from about 200 Da to about 60,000 Da, any of which may used for the purposes of the present disclosure. In certain embodiments, PEGs having molecular weights from 200 Da to 2,000 Da or from 200 Da to 500 Da are used. Different forms of PEG may also be used, depending on the initiator used for the polymerization process; a common initiator is a monofunctional methyl ether PEG, or methoxypoly(ethylene glycol), abbreviated mPEG. Lower-molecular-weight PEGs are also available as pure oligomers, referred to as monodisperse, uniform, or discrete. These are used in certain embodiments of the present disclosure.

PEGs are also available with different geometries: branched PEGs have three to ten PEG chains emanating from a central core group; star PEGs have 10 to 100 PEG chains emanating from a central core group; and comb PEGs have multiple PEG chains normally grafted onto a polymer backbone. PEGs can also be linear. The numbers that are often included in the names of PEGs indicate their average molecular weights (e.g., a PEG with n=9) would have an average molecular weight of approximately 400 daltons, and would be labeled PEG 400.

As used herein, “PEGylation” is the act of covalently coupling a PEG structure to the peptide inhibitor of the invention, which is then referred to as a “PEGylated peptide inhibitor”. In certain embodiments, the PEG of the PEGylated side chain is a PEG with a molecular weight from about 200 Da to about 40,000 Da.

In various embodiments, the agents are present in a pharmaceutical composition comprising one or more pharmaceutically acceptable diluents, carriers, or excipients. A pharmaceutically acceptable carrier, diluent or excipient refers to a non-toxic solid, semi-solid or liquid filler, diluent, encapsulating material or formulation auxiliary of any type. The term “pharmaceutically acceptable carrier” includes any of the standard pharmaceutical carriers. Pharmaceutically acceptable carriers for therapeutic use are well known in the pharmaceutical art and are described, for example, in “Remington's Pharmaceutical Sciences”, 17th edition, Alfonso R. Gennaro (Ed.), Mark Publishing Company, Easton, PA, USA, 1985. For example, sterile saline and phosphate-buffered saline at slightly acidic or physiological pH may be used. Suitable pH-buffering agents may, e.g., be phosphate, citrate, acetate, tris(hydroxymethyl)aminomethane (TRIS), N-tris(hydroxymethyl)methyl-3-aminopropanesulfonic acid (TAPS), ammonium bicarbonate, diethanolamine, histidine, arginine, lysine or acetate (e.g., as sodium acetate), or mixtures thereof. The term further encompasses any carrier agents listed in the US Pharmacopeia for use in animals, including humans.

### EXAMPLES

The following examples demonstrate certain specific embodiments of the present invention. The following examples were carried out using standard techniques that are well known and routine to those of skill in the art, except where otherwise described in detail. It is to be understood that these examples are for illustrative purposes only and do not purport to be wholly definitive as to conditions or scope of the invention. As such, they should not be construed in any way as limiting the scope of the present invention.

### Abbreviations

DCM: dichloromethane
DMF: N,N-dimethylformamide
NMP: N-methylpyrolidone
HBTU: O-(Benzotriazol-1-yl)-N,N,N′,N′-tetramethyluronium hexafluorophosphate
HATU: 2-(7-aza-1H-benzotriazole-1-yl)-1,1,3,3-tetramethyluronium
hexafluorophosphate
DCC: Dicyclohexylcarbodiimide
NHS: N-hydoxysuccinimide
DIPEA: diisopropylethylamine
EtOH: ethanol
Et2O: diethyl ether
Hy: hydrogen
TFA: trifluoroacetic acid
TIS: triisopropylsilane
ACN: acetonitrile
HPLC: high performance liquid chromatography
ESI-MS: electron spray ionization mass spectrometry
PBS: phosphate-buffered saline
Boc: t-butoxycarbonyl
Fmoc: Fluorenylmethyloxycarbonyl
Acm: acetamidomethyl
IVA: Isovaleric acid (or Isovaleryl)

K( ): In the peptide sequences provided herein, wherein a compound or chemical group is presented in parentheses directly after a Lysine residue, it is to be understood that the compound or chemical group in the parentheses is a side chain conjugated to the Lysine residue. So, e.g., but not to be limited in any way, K-[(PEG8)]- indicates that a PEG8 moiety is conjugated to a side chain of this Lysine.

Palm: Indicates conjugation of a palmitic acid (palmitoyl).

As used herein “C( )” refers to a cysteine residue involved in a particular disulfide bridge. For example, in Hepcidin, there are four disulfide bridges: the first between the two C(1) residues; the second between the two C(2) residues; the third between the two C(3) residues; and the fourth between the two C(4) residues. Accordingly, in some embodiments, the sequence for Hepcidin is written as follows:

(SEQ ID NO: 66)
Hy-DTHFPIC(1)IFC(2)C(3)GC(2)C(4)HRSKC(3)GMC(4)C(1)
KT-OH;

and the sequence for other peptides may also optionally be written in the same manner.

### Example 1

### Synthesis of Peptide Mimetics

Unless otherwise specified, reagents and solvents employed in the following were available commercially in standard laboratory reagent or analytical grade, and were used without further purification.

### Procedure for Solid-Phase Synthesis of Peptides

Peptide mimetics of the invention were chemically synthesized using optimized 9-fluorenylmethoxy carbonyl (Fmoc) solid phase peptide synthesis protocols. For C-terminal amides, rink-amide resin was used, although wang and trityl resins were also used to produce C-terminal acids. The side chain protecting groups were as follows: Glu, Thr and Tyr: 0-tButyl; Trp and Lys: t-Boc (t-butyloxycarbonyl); Arg: N-gamma-2,2,4,6,7-pentamethyldihydrobenzofuran-5-sulfonyl; His, Gln, Asn, Cys: Trityl. For selective disulfide bridge formation, Acm (acetamidomethyl) was also used as a Cys protecting group. For coupling, a four to ten-fold excess of a solution containing Fmoc amino acid, HBTU and DIPEA (1:1:1.1) in DMF was added to swelled resin [HBTU: 0-(Benzotriazol-1-yl)-N,N,N′,N′-tetramethyluronium hexafluorophosphate; DIPEA: diisopropylethylamine; DMF: dimethylformamide]. HATU (0-(7-azabenzotriazol-1-yl)-1,1,3,3,-tetramethyluronium hexafluorophosphate) was used instead of HBTU to improve coupling efficiency in difficult regions. Fmoc protecting group removal was achieved by treatment with a DMF, piperidine (2:1) solution.

### Procedure for Cleavage of Peptides Off Resin

Side chain deprotection and cleavage of the peptide mimetics (e.g., Compound No. 2) was achieved by stirring dry resin in a solution containing trifluoroacetic acid, water, ethanedithiol and tri-isopropylsilane (90:5:2.5:2.5) for 2 to 4 hours. Following TFA removal, peptide was precipitated using ice-cold diethyl ether. The solution was centrifuged and the ether was decanted, followed by a second diethyl ether wash. The peptide was dissolved in an acetonitrile, water solution (1:1) containing 0.1% TFA (trifluoroacetic acid) and the resulting solution was filtered. The linear peptide quality was assessed using electrospray ionization mass spectrometry (ESI-MS).

### Procedure for Purification of Peptides

Purification of the peptides of the invention (e.g., Compound No. 2) was achieved using reverse-phase high performance liquid chromatography (RP-HPLC). Analysis was performed using a C18 column (3 μm, 50×2 mm) with a flow rate of 1 mL/min. Purification of the linear peptides was achieved using preparative RP-HPLC with a C18 column (5 μm, 250×21.2 mm) with a flow rate of 20 mL/min. Separation was achieved using linear gradients of buffer B in A (Buffer A: Aqueous 0.05% TFA; Buffer B: 0.043% TFA, 90% acetonitrile in water).

### Procedure for Oxidation of Peptides.

Method A (Single disulfide oxidation). Oxidation of the unprotected peptides of the invention was achieved by adding drop-wise iodine in MeOH (1 mg per 1 mL) to the peptide in a solution (ACN: H₂O, 7:3, 0.5% TFA). After stirring for 2 min, ascorbic acid portion wise was added until the solution was clear and the sample was immediately loaded onto the HPLC for purification.

Method B (Selective oxidation of two disulfides). When more than one disulfide was present, selective oxidation was often performed. Oxidation of the free cysteines was achieved at pH 7.6 NH₄CO₃ solution at 1 mg/10 mL of peptide. After 24 h stirring and prior to purification the solution was acidified to pH 3 with TFA followed by lyophilization. The resulting single oxidized peptides (with ACM protected cysteines) were then oxidized/selective deprotection using iodine solution. The peptide (1 mg per 2 mL) was dissolved in MeOH/H₂O, 80:20 iodine dissolved in the reaction solvent was added to the reaction (final concentration: 5 mg/mL) at room temperature. The solution was stirred for 7 minutes before ascorbic acid was added portion wise until the solution is clear. The solution was then loaded directly onto the HPLC.

Method C (Native oxidation). When more than one disulfide was present and when not performing selective oxidations, native oxidation was performed. Native oxidation was achieved with 100 mM NH₄CO₃ (pH7.4) solution in the presence of oxidized and reduced glutathione (peptide/GSH/GSSG, 1:100:10 molar ratio) of (peptide: GSSG: GSH, 1:10, 100). After 24 h stirring and prior to RP-HPLC purification the solution was acidified to pH 3 with TFA followed by lyophilization.

Procedure of cysteine oxidation to produce dimers. Oxidation of the unprotected peptides of the invention was achieved by adding drop-wise iodine in MeOH (1 mg per 1 mL) to the peptide in a solution (ACN: H₂O, 7:3, 0.5% TFA). After stirring for 2 min, ascorbic acid portion wise was added until the solution was clear and the sample was immediately loaded onto the HPLC for purification.

### Procedure for Dimerization.

Glyoxylic acid (DIG), IDA, or Fmoc-β-Ala-IDA was pre-activated as the N-hydoxysuccinimide ester by treating 1 equivalent (abbreviated “eq”) of the acid with 2.2 eq of both N-hydoxysuccinimide (NHS) and dicyclohexyl carbodiimide (DCC) in NMP (N-methyl pyrolidone) at a 0.1 M final concentration. For the PEG13 and PEG25 linkers, these chemical entities were purchased pre-formed as the activated succinimide ester. The activated ester ˜ 0.4 eq was added slowly to the peptide in NMP (1 mg/mL) portionwise. The solution was left stirring for 10 min before 2-3 additional aliquots of the linker ˜0.05 eq were slowly added. The solution was left stirring for a further 3 h before the solvent was removed under vaccuo and the residue was purified by reverse phase HPLC. An additional step of stirring the peptide in 20% piperidine in DMF (2×10 min) before an additional reverse phase HPLC purification was performed.

One of skill in the art will appreciate that standard methods of peptide synthesis may be used to generate the compounds of the invention.

### Linker Activation and Dimerization

Peptide monomer subunits were linked to form hepcidin mimetic peptide dimers as described below.

Small Scale DIG Linker Activation Procedure: 5 mL of NMP was added to a glass vial containing IDA diacid (304.2 mg, 1 mmol), N-hydroxysuccinimide (NHS, 253.2 mg, 2.2 eq. 2.2 mmol) and a stirring bar. The mixture was stirred at room temperature to completely dissolve the solid starting materials. N, N′-Dicyclohexylcarbodiimide (DCC, 453.9 mg, 2.2 eq., 2.2 mmol) was then added to the mixture. Precipitation appeared within 10 min and the reaction mixture was further stirred at room temperature overnight. The reaction mixture was then filtered to remove the precipitated dicyclohexylurea (DCU). The activated linker was kept in a closed vial prior to use for dimerization. The nominal concentration of the activated linker was approximately 0.20 M.

For dimerization using PEG linkers, there was no pre-activation step involved. Commercially available pre-activated bi-functional PEG linkers were used.

Dimerization Procedure: 2 mL of anhydrous DMF was added to a vial containing peptide monomer (0.1 mmol). The pH of the peptide was the adjusted to 8-9 with DIEA. Activated linker (IDA or PEG13, PEG 25) (0.48eq relative to monomer, 0.048 mmol) was then added to the monomer solution. The reaction mixture was stirred at room temperature for one hour. Completion of the dimerization reaction was monitored using analytical HPLC. The time for completion of dimerization reaction varied depending upon the linker. After completion of reaction, the peptide was precipitated in cold ether and centrifuged. The supernatant ether layer was discarded. The precipitation step was repeated twice. The crude dimer was then purified using reverse phase HPLC (Luna C18 support, 10u, 100A, Mobile phase A: water containing 0.1% TFA, mobile phase B: Acetonitrile (ACN) containing 0.1% TFA, gradient of 15% B and change to 45% B over 60 min, flow rate 15 ml/min). Fractions containing pure product were then freeze-dried on a lyophilizer.

### Conjugation of Half-Life Extension Moieties

Conjugation of peptides were performed on resin. Lys(ivDde) was used as the key amino acid. After assembly of the peptide on resin, selective deprotection of the ivDde group occurred using 3×5 min 2% hydrazine in DMF for 5 min. Activation and acylation of the linker using HBTU, DIEA 1-2 equivalents for 3 h, and Fmoc removal followed by a second acylation with the lipidic acid gave the conjugated peptide.

### Example 2

### Efficacy Study of Hepcidin Mimetics in a Mouse Model of Sickle Cell Disease

### Experimental Methods

Compound 46, a hepcidin mimetic, was administered subcutaneously at 1.0 mg/kg (group 3, N=9) or 2.5 mg/kg (group 4, N=9) in Townes SCD mice. 36 Townes SCD and 6 129S1/SvlmJ (wild-type), female mice, 5 weeks old upon delivery. A control group of wild-type mice (groups 1, N=6) was treated with vehicle as well as another SCD mice (group 2, N=9). All mice were dosed three times per week (TIW). All mice were maintained on an iron-adjusted diet (TD.140258, 35 ppm iron) throughout the study. Collections and necropsies were performed 24 hours post-last dose of compound or vehicle. After 4 weeks on study, animals from each group were euthanized, EDTA whole blood collected for CBC and serum was collected for hemolysis biomarker analysis. Spleens and livers were weighed at terminus. Liver, spleen, kidney, heart and brain were collected for future analysis. Serum and urine were collected to analyze chemistry markers, such as bilirubin, LDH (lactate dehydrogenase), haptoglobin, urine creatinine, urine total protein and urine urea nitrogen.

### Spleen Weights and Spleen Body Weight Ratio

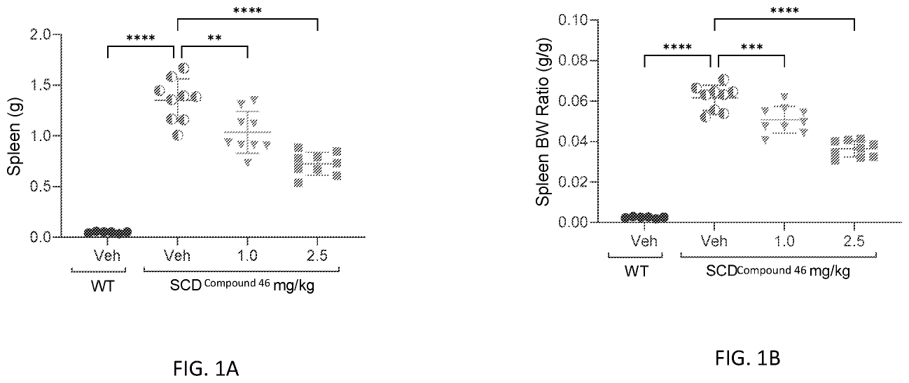

As shown in FIG. 1A and FIG. 1B, spleen size was significantly larger in SCD mice compare to healthy control mice. Spleen weights and spleen to body weight ratios were significantly and dose-dependently lower in SCD mice treated with a hepcidin mimetic as disclosed herein, such as compound 46, compared to vehicle.

### Liver Weights and Liver Body Weight Ratio

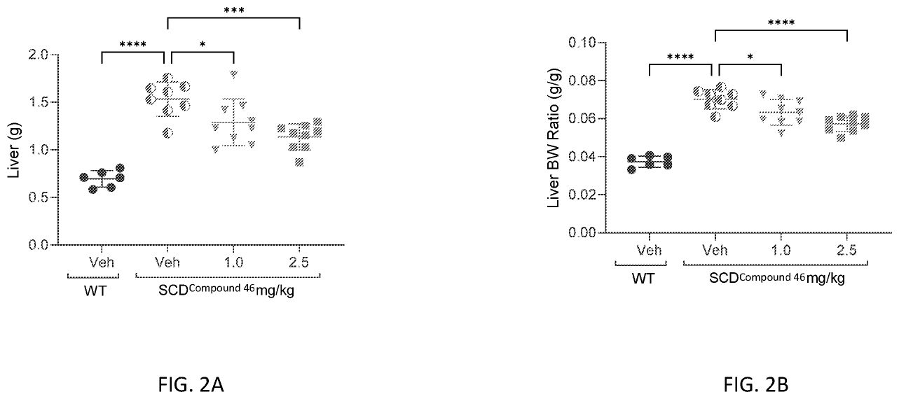

As shown in FIG. 2A and FIG. 2B, liver size was significantly enlarged in SCD mice compare to that in healthy control mice. Compared to vehicle treated SCD mice, liver weight and liver to body weight ratios of SCD mice treated with a hepcidin mimetic as disclosed herein, such as compound 46, were significantly lower in a dose dependent manner.

### Red Blood Cell and Hemoglobin

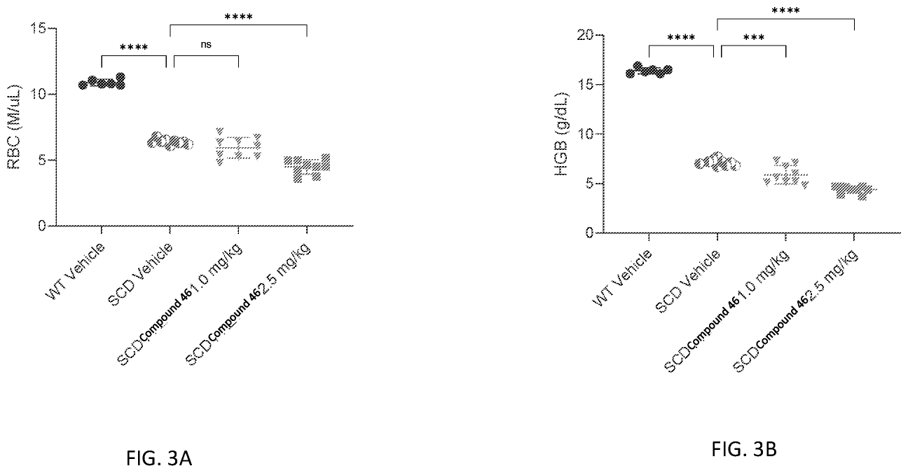

As shown in FIG. 3A and FIG. 3B, red blood cell (RBC) count and Hgb are significantly lower in SCD mice compared to healthy control mice. RBC count is significantly reduced with 2.5 mg/kg dose of a hepcidin mimetic as disclosed herein, such as compound 46. Hgb level is significantly reduced in all three dose groups, especially in the high dose group.

### Reticulocyte Counts

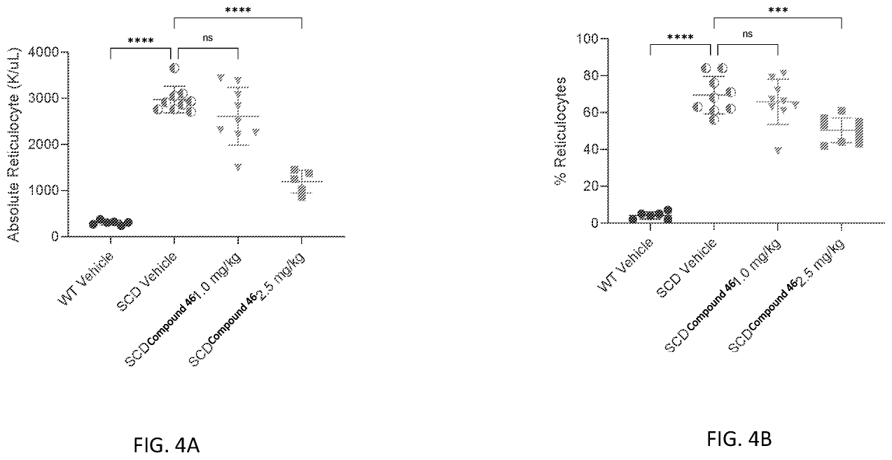

As shown in FIG. 4A and FIG. 4B, reticulocytes were counted both with the automated analyzer as well as manually from a new methylene blue stained blood smear by counting the number of positive stained cells encountered in 500 erythrocytes and calculating the percentage. Percent of reticulocytes were significantly higher in SCD mice compared to in healthy control. A hepcidin mimetic as disclosed herein, such as compound 46, at 2.5 mg/kg dose decreased percent of reticulocytes significantly compared to vehicle control

### Hematocrit and Mean Corpuscular Volume

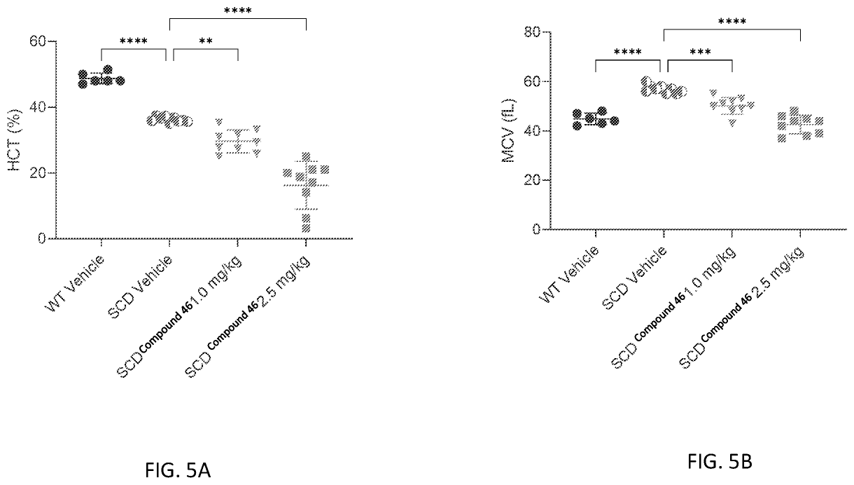

As shown in FIG. 5A and FIG. 5B, hematocrit_(HCT) was significantly decreased in SCD mice compared to healthy control mice. Treatment of SCD mice with a hepcidin mimetic disclosed herein, such as compound 46, resulted in a significant reduction of HCT in a dose dependent manner. Mean corpuscular volume (MCV) was significantly increased in SCD mice compared to that in healthy control mice. Treatment of SCD mice with a hepcidin mimetic disclosed herein, such as compound 46, resulted in a significant reduction of MCV in a dose dependent manner as compared to the vehicle group.

### Mean Corpuscular Hemoglobin and Mean Corpuscular Hemoglobin Concentration

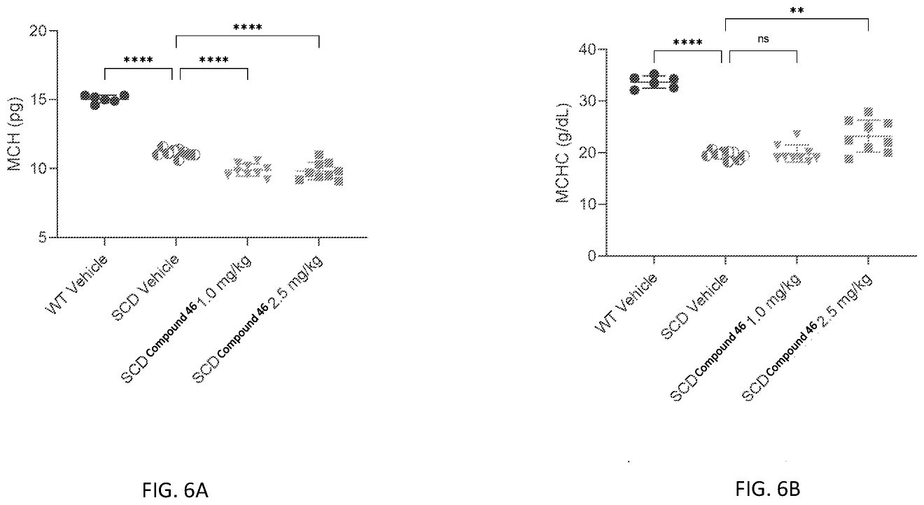

As shown in FIG. 6A and FIG. 6B, both mean corpuscular hemoglobin (MCH) and mean corpuscular hemoglobin concentration (MCHC) were significantly lower in SCD mice compared to healthy control mice. Treatment of SCD mice with a hepcidin mimetic disclosed herein, such as compound 46, resulted in a significant reduction in MCH compared to vehicle control. Treatment of SCD mice with a hepcidin mimetic disclosed herein, such as 2.5 mg/kg compound 46, resulted in a significant increase in MCHC compared to vehicle control.

### White Blood Cell and Lymphocyte Count

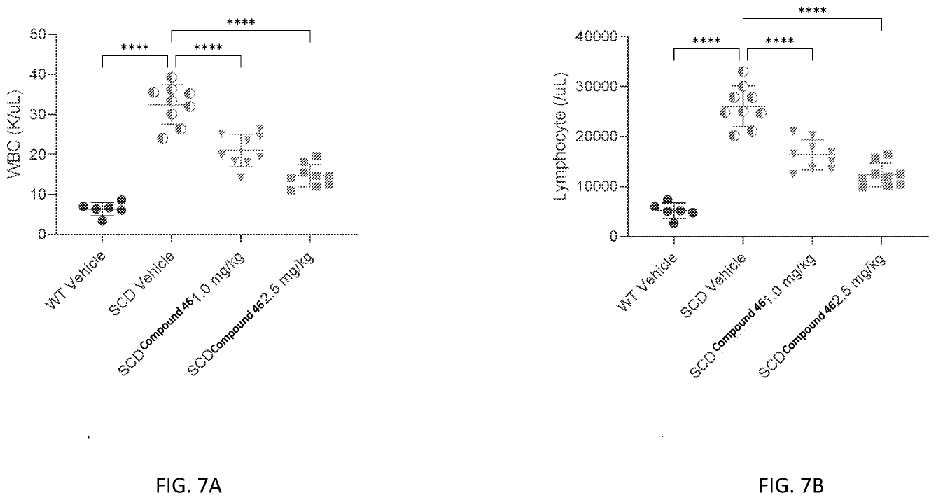

As shown in FIG. 7A and FIG. 7B, white blood cell (WBC) and lymphocyte counts were significantly increased in the SCD mice compared to those in healthy control. Treatment of SCD mice with a hepcidin mimetic disclosed herein, such as compound 46, resulted in the significant WBC and lymphocyte reduction in a dose dependent manner.

### Neutrophil and Monocytes Count

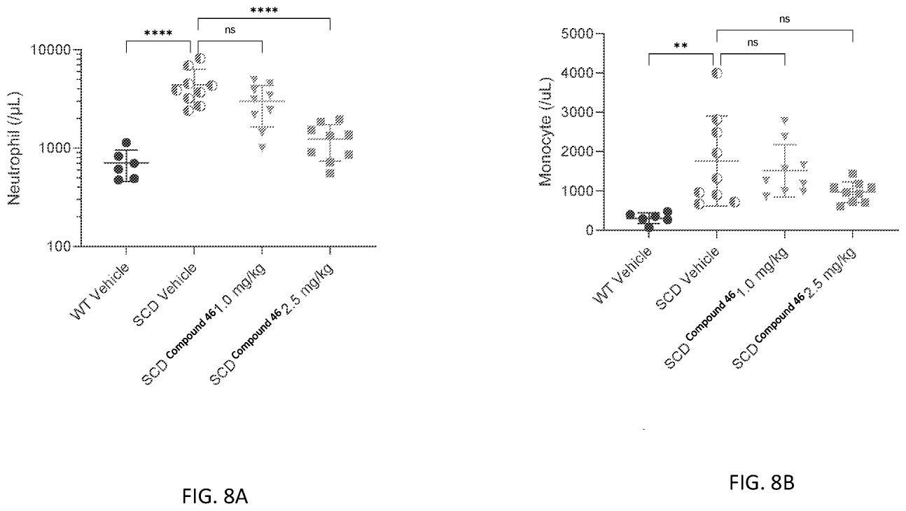

As shown in FIG. 8A and FIG. 8B, neutrophil and monocyte count were significantly increased in SCD mice compared to those in healthy control. Treatment of SCD mice with a hepcidin mimetic disclosed herein, such as compound 46, significantly decreased neutrophil count in a dose dependent manner.

### Serum Total Bilirubin and Serum Lactate Dehydrogenase

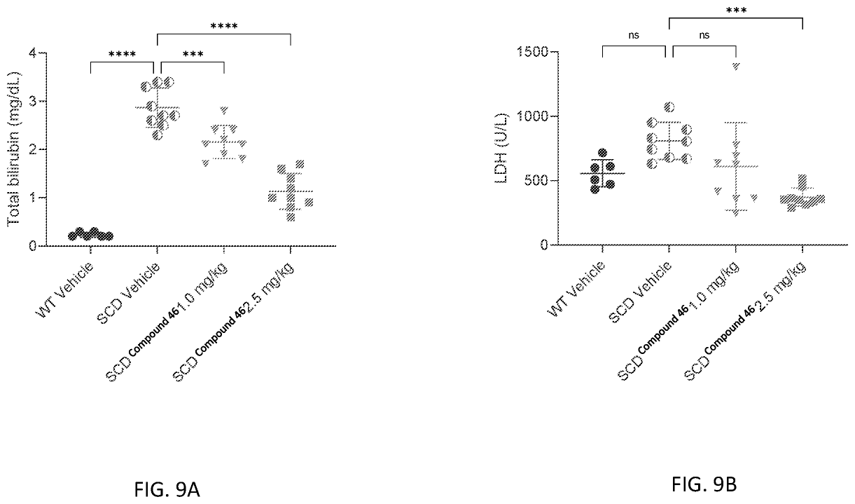

Serum total bilirubin and serum lactate dehydrogenase are hemolysis biomarkers. As shown in FIG. 9A and FIG. 9B, serum lactate dehydrogenase (LDH) and total bilirubin were significantly higher in SCD mice compared to those in healthy control, which reflect extent of hemolysis. Treatment of SCD mice with a hepcidin mimetic disclosed herein, such as compound 46, significantly reduced both parameters in a dose dependent manner.

Hepcidin mimetic peptide may therefore be potentially beneficial in lowering mean corpuscular hemoglobin concentration and alleviate RBC sickling, thereby improving hemodynamics and oxygen carrying capacity, and preventing hemolysis and vaso-occlusion.

Table 1 summarizes hematological parameters obtained from the treatment studies.

TABLE 1
Summary of Hematological Parameters

| | Control + Vehicle | SCD + Vehicle | SCD + Compound 46 | |
|---|---|---|---|---|
| Dose | N/A | N/A | 1 | 2.5 |
| RBC (M/uL) | 10.89 ± 0.25 **** | 6.39 ± 0.20 | 5.94 ± 0.78 | 4.49 ± 0.55 **** |
| HCT (%) | 48.73 ± 1.63 **** | 36.26 ± 0.82 | 29.62 ± 3.45 ** | 16.27 ± 7.23 **** |
| HGB (g/dL) | 16.40 ± 0.30 **** | 7.10 ± 0.32 | 5.89 ± 0.93 *** | 4.39 ± 0.36 **** |
| Abs Retics (K/uL) | 302.7 ± 47.03 **** | 2973 ± 288.3 | 2612 ± 627.9 | 1195 ± 246.5 **** |
| % Retics (%) | 4.17 ± 1.94 **** | 69.44 ± 10.13 | 65.78 ± 12.23 | 50.33 ± 6.73 *** |
| MCV (fL) | 44.83 ± 2.32 | 56.89 ± 1.62 | 50.11 ± 3.41 | 42.56 ± 3.81 |
| MCH (pg) | 15.05 ± 0.27 **** | 11.13 ± 0.29 | 9.89 ± 0.45 ** | 9.82 ± 0.63 ** |
| MCHC (g/dL) | 33.68 ± 1.18 **** | 19.59 ± 0.78 | 19.82 ± 1.65 | 23.22 ± 3.10 |

* P < 0.05,
** P < 0.01,
*** P < 0.001,
**** P < 0.0001 vs SCD + Vehicle

Table 2 summarizes serum chemistry results for the treatment studies.

TABLE 2
Summary of serum chemistry

| | Control + Vehicle | SCD + Vehicle | SCD + Compound 46 | |
|---|---|---|---|---|
| Dose | N/A | N/A | 1 | 2.5 |
| Total bilirubin (mg/dL) | 0.23 ± 0.05 **** | 2.87 ± 0.41 | 2.16 ± 0.34 **** | 1.13 ± 0.37 **** |
| LDH (U/L) | 556.5 ± 105.2 * | 809.2 ± 145.3 | 610.6 ± 341.0 | 372.3 ± 70.96 **** |

* P < 0.05,
** P < 0.01,
*** P < 0.001,
**** P < 0.0001 vs SCD + Vehicle

Table 3 shows data for spleen weights, liver weights, spleen to body weight ratios and liver to body weight ratios.

TABLE 3
Spleen or Liver Weights and Ratios of Spleen or Liver Weight versus Body Weight

| | Control + vehicle | SCD + vehicle | SCD + Compound 46 | | |
|---|---|---|---|---|---|
| Dose | N/A | N/A | 1 | 2.5 | 7.5 |
| Spleen | 0.048 ± | 1.353 ± | 1.035 ± | 0.725 ± | 0.301 ± |
| Weight (g) | 0.001 **** | 0.211 | 0.207 *** | 0.113 **** | 0.043 **** |
| Spleen:BW | 0.0026 ± | 0.0616 ± | 0.0508 ± | 0.0365 ± | 0.0217 ± |
| Ratio (g/g) | 0.0001 **** | 0.0063 | 0.0066 **** | 0.0040 **** | 0.0018 **** |
| Liver | 0.070 ± | 1.534 ± | 1.290 ± | 1.138 ± | 0.758 ± |
| Weight (g) | 0.087 **** | 0.182 | 0.244 * | 0.135 **** | 0.093 **** |
| Liver:BW | 0.0374 ± | 0.0702 ± | 0.0633 ± | 0.0573 ± | 0.0549 ± |
| Ratio (g/g) | 0.0029 **** | 0.0050 | 0.0068 * | 0.0041 **** | 0.0037 **** |

* P < 0.05,
** P < 0.01,
*** P < 0.001,
**** P < 0.0001 vs SCD + Vehicle

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

1. A method for treating sickle cell disease in a subject, the method comprising administering to the subject an effective amount of a hepcidin mimetic or a pharmaceutically acceptable salt thereof or a pharmaceutical composition comprising a hepcidin mimetic or a pharmaceutically acceptable salt thereof and a pharmaceutically acceptable excipient or carrier.

2. The method of claim 1, wherein hepcidin mimetic is a peptide comprising or having Formula I:
R1-X—Y—R2 (I)
or a pharmaceutically acceptable salt or solvate thereof,
wherein
R1 is hydrogen, a C1-C6 alkyl, a C6-C12 aryl, a C1-C20 alkanoyl, or pGlu;
R2 is NH₂ or OH;
X is an amino acid sequence of Formula II:
X1-X2-X3-X4-X5-X6-X7-X8-X9-X10 (II)
wherein
X1 is Asp, Ala, Ida, pGlu, bhAsp, Leu, D-Asp, or absent;
X2 is Thr, Ala, or D-Thr;
X3 is His, Lys, D-His, or Lys;
X4 is Phe, Ala, Dpa, or D-Phe;
X5 is Pro, Gly, Arg, Lys, Ala, D-Pro, or bhPro;
X6 is Ile, Cys, Arg, Lys, D-Ile, or D-Cys;
X7 is Cys, Ile, Leu, Val, Phe, D-Ile, or D-Cys;
X8 is Ile, Arg, Phe, Gln, Lys, Glu, Val, Leu, or D-Ile;
X9 is Phe or bhPhe; and
X10 is Lys, Phe, or absent;
wherein if Y is absent, X7 is Ile; and
Y is an amino acid sequence of Formula III:
Y1-Y2-Y3-Y4-Y5-Y6-Y7-Y8-Y9-Y10-Y11-Y12-Y13-Y14-Y15 (III)
wherein
Y1 is Gly, Cys, Ala, Phe, Pro, Glu, Lys, D-Pro, Val, Ser, or absent;
Y2 is Pro, Ala, Cys, Gly, or absent;
Y3 is Arg, Lys, Pro, Gly, His, Ala, Trp, or absent;
Y4 is Ser, Arg, Gly, Trp, Ala, His, Tyr, or absent;
Y5 is Lys, Met, Arg, Ala, or absent;
Y6 is Gly, Ser, Lys, Ile, Ala, Pro, Val, or absent;
Y7 is Trp, Lys, Gly, Ala, Ile, Val, or absent;
Y8 is Val, Thr, Gly, Cys, Met, Tyr, Ala, Glu, Lys, Asp, Arg, or absent;
Y9 is Cys, Tyr, or absent;
Y10 is Met, Lys, Arg, Tyr, or absent;
Y11 is Arg, Met, Cys, Lys, or absent;
Y12 is Arg, Lys, Ala or absent;
Y13 is Arg, Cys, Lys, Val or absent;
Y14 is Arg, Lys, Pro, Cys, Thr or absent; and
Y15 is Thr, Arg or absent;
wherein the peptide of Formula I is optionally PEGylated on R1, X, or Y;
wherein a side chain of an amino acid of the peptide is optionally conjugated to a lipophilic substituent or polymeric moiety;
wherein the peptide of Formula I optionally has a disulfide bond formed between the thiol groups of two cysteine residues in the same peptide: and
wherein Ida is iminodiacetic acid, pGlu is pyroglutamic acid, bhAsp is β-homoaspartic acid, and bhPro is β-homoproline.

3. The method of claim 2, wherein R1 is hydrogen, isovaleric acid, isobutyric acid or acetyl.

4. The method of claim 2, wherein X comprises or consists of an amino acid sequence of Formula IV:
X1-Thr-His-X4-X5-X6-X7-X8-Phe-X10 (IV)
wherein
X1 is Asp, Ida, pGlu, bhAsp or absent;
X4 is Phe or Dpa;
X5 is Pro or bhPro;
X6 is Ile, Cys, or Arg;
X7 is Cys, Ile, Leu, or Val;
X8 is Ile, Lys, Glu, Phe, Gln, or Arg; and
X10 is Lys or absent.

5. The method of claim 2, wherein X comprises or consists of an amino acid sequence of Formula V:
X1-Thr-His-X4-X5-Cys-Ile-X8-Phe-X10 (V)
wherein
X1 is Asp, Ida, pGlu, bhAsp, or absent;
X4 is Phe or Dpa;
X5 is Pro or bhPro;
X8 is Ile, Lys, Glu, Phe, Gln or Arg; and
X10 is Lys or absent.

6. The method of claim 2, wherein the peptide has Formula VI:
R¹—X—Y—R² (VI)
or a pharmaceutically acceptable salt thereof, wherein:
R¹ is hydrogen, isovaleric acid, isobutyric acid, or acetyl;
R² is NH₂ or OH;
X is an amino acid sequence of Formula VII:
X1-Thr-His-X4-X5-Cys-Ile-X8-Phe-X10 (VII)
wherein
X1 is Asp, Ida, pGlu, bhAsp, or absent;
X4 is Phe or Dpa;
X5 is Pro or bhPro;
X8 is Ile, Lys, Glu, Phe, Gln, or Arg; and
X10 is Lys or absent;
wherein Y is an amino acid sequence of Formula VIII:
Y1-Pro-Y3-Ser-Y5-Y6-Y7-Y8-Cys-Y10 (VIII)
wherein
Y1 is Gly, Glu, Val, or Lys;
Y3 is Arg or Lys;
Y5 is Arg or Lys;
Y6 is Gly, Ser, Lys, Ile, or Arg;
Y7 is Trp or absent;
Y8 is Val, Thr, Asp, Glu, or absent; and
Y10 is Lys or absent;
wherein the peptide has a disulfide bond formed between the thiol groups of two cysteine residues in the same peptide;
wherein the peptide is optionally PEGylated on R¹, X, or Y;
wherein a side chain of an amino acid of the peptide is optionally conjugated to a lipophilic substituent or polymeric moiety; and
wherein Ida is iminodiacetic acid; pGlu is pyroglutamic acid; bhAsp is β-homoaspartic acid; and bhPro is β-homoproline.

7. The method of claim 1, wherein the hepcidin mimetic is a peptide comprising a sequence or structure selected from:
(SEQ ID NO: 47)
DTHFPICIFGPRSKGWVC;
(SEQ ID NO: 48)
DTHFPCIIFGPRSKGWVCK;
(SEQ ID NO: 49)
DTHFPCIIFEPRSKGWVCK;
(SEQ ID NO: 50)
DTHFPCIIFGPRSKGWACK;
(SEQ ID NO: 51)
DTHFPCIIFGPRSKGWVCKK;
(SEQ ID NO: 52)
DTHFPCIIFVCHRPKGCYRRVCR;
(SEQ ID NO: 53)
DTHFPCIKFGPRSKGWVCK;
(SEQ ID NO: 54)
DTHFPCIKFKPRSKGWVCK;
(SEQ ID NO: 55)
DTHFPCIIFGPRSRGWVCK;
(SEQ ID NO: 56)
DTHFPCIKFGPKSKGWVCK;
(SEQ ID NO: 57)
DTHFPCIKFEPRSKGCK;
(SEQ ID NO: 58)
DTHFPCIKFEPKSKGWECK;
(SEQ ID NO: 59)
DTHFPCIKFEPRSKKCK;
(SEQ ID NO: 60)
DTHFPCIKFEPRSKGCKK;
(SEQ ID NO: 61)
DTHFPCIKFKPRSKGCK;
(SEQ ID NO: 62)
DTHFPCIKFEPKSKGCK;
(SEQ ID NO: 63)
DTHFPCIKF;
(SEQ ID NO: 64)
DTHFPCIIF;
and
(SEQ ID NO: 65)
DTKFPCIIF,
or a pharmaceutically acceptable salt thereof,
wherein the peptide is optionally PEGylated on R1, X, or Y; and
wherein a side chain of an amino acid of the peptide is optionally conjugated to a lipophilic substituent or polymeric moiety.

8. The method of claim 1, wherein the hepcidin mimetic is a peptide comprising a sequence or structure selected from:
(Compound 1; SEQ ID NO: 1)
Isovaleric acid-DTHFPICIFGPRSKGWVC-NH₂;
(Compound 2; SEQ ID NO: 2)
Isovaleric acid-DTHFPCIIFGPRSKGWVCK-NH₂;
(Compound 3; SEQ ID NO: 3)
Isovaleric acid-DTHFPCIIFEPRSKGWVCK-NH₂;
(Compound 4; SEQ ID NO: 4)
Isovaleric acid-DTHFPCIIFGPRSKGWACK-NH₂;
(Compound 5; SEQ ID NO: 5)
Isovaleric acid-DTHFPCIIFGPRSKGWVCKK-NH₂;
(Compound 6; SEQ ID NO: 6)
Isovaleric acid-DTHFPCIIFVCHRPKGCYRRVCR-NH₂;
(Compound 7; SEQ ID NO: 7)
Isovaleric acid-DTHFPCI(K(PEG8))FGPRSKGWVCK-NH₂;
(Compound 8; SEQ ID NO: 8)
Isovaleric acid-DTHFPCIKF(K(PEG8))PRSKGWVCK-NH₂;
(Compound 9; SEQ ID NO: 9)
Isovaleric acid-DTHFPICIFGPRS(K(PEG8))GWVC-NH₂;
(Compound 10; SEQ ID NO: 10)
Isovaleric acid-DTHFPICIFGPRS(K(PEG4))GWVC-NH₂;
(Compound 11; SEQ ID NO: 11)
Isovaleric acid-DTHFPCIIFGPRSRGWVC(K(PEG8))-NH₂;
(Compound 12; SEQ ID NO: 12)
Isovaleric acid-DTHFPCIIFGPRSRGWVC(K(PEG4))-NH₂;
(Compound 13; SEQ ID NO: 13)
Isovaleric acid-DTHFPCIIFGPRSRGWVC(K(PEG2))-NH₂;
(Compound 14; SEQ ID NO: 14)
Isovaleric acid-DTHFPCI(K(Palm))FGPRSKGWVCK-NH₂;
(Compound 15; SEQ ID NO: 15)
Isovaleric acid-DTHFPCIKF)K(Palm))PRSKGWVCK-NH₂;
(Compound 16; SEQ ID NO: 16)
Isovaleric acid-DTHFPCIKFGP(K(Palm))SKGWVCK-NH₂;
(Compound 17; SEQ ID NO: 17)
Isovaleric acid-DTHFPCIKFGPRS(K(Palm))GWVCK-NH₂;
(Compound 18; SEQ ID NO: 18)
Isovaleric acid-DTHFPCIKFGPRSKGWVC(K(Palm))NH₂;
(Compound 19; SEQ ID NO: 19)
Isovaleric acid-DTHFPCI(K(PEG3-Palm))FGPRSKGWVCK-NH₂;
(Compound 20; SEQ ID NO: 20)
Isovaleric acid-DTHFPCIKF(K(PEG3-Palm))PRSKGWVCK-NH₂;
(Compound 21; SEQ ID NO: 21)
Isovaleric acid-DTHFPCIKFGP(K(PEG3-Palm))SKGWVCK-NH₂;
(Compound 22; SEQ ID NO: 22)
Isovaleric acid-DTHFPCIKFGPRS(K(PEG3-Palm))GWVCK-NH₂;
(Compound 23; SEQ ID NO: 23)
Isovaleric acid-DTHFPCIKFGPRSKGWVC(K(PEG3-Palm))-NH₂;
(Compound 24; SEQ ID NO: 24)
Isovaleric acid-DTHFPCIKFGPRSKGWVC(K(PEG8))-NH₂;
(Compound 25; SEQ ID NO: 25)
Isovaleric acid-DTHFPCI(K(isoGlu-Palm))FEPRSKGCK-NH₂;
(Compound 26; SEQ ID NO: 26)
Isovaleric acid-DTHFPCIKF-K(isoGlu-Palm)-PRSKGCK-NH₂;
(Compound 27; SEQ ID NO: 27)
Isovaleric acid-DTHFPCIKFEP(K(isoGlu-Palm))SKGCK-NH₂;
(Compound 28; SEQ ID NO: 28)
Isovaleric acid-DTHFPCIKFEP(K(isoGlu-Palm))SKGWECK-NH₂;
(Compound 29; SEQ ID NO: 29)
Isovaleric acid-DTHFPCIKFEPRS(K(isoGlu-Palm))GCK-NH₂;
(Compound 30; SEQ ID NO: 30)
Isovaleric acid-DTHFPCIKFEPRSK(K(isoGlu-Palm))CK-NH₂;
(Compound 31; SEQ ID NO: 31)
Isovaleric acid-DTHFPCIKFEPRSKGCK(K(isoGlu-Palm))-NH₂;
(Compound 32; SEQ ID NO: 32)
Isovaleric acid-DTHFPCI-K(Dapa-Palm)-FEPRSKGCK-NH₂;
(Compound 33; SEQ ID NO: 33)
Isovaleric acid-DTHFPCIK(F(Dapa-Palm))PRSKGCK-NH₂;
(Compound 34; SEQ ID NO: 34)
Isovaleric acid-DTHFPCIKFEP(K(Dapa-Palm))SKGCK-NH₂;
(Compound 35; SEQ ID NO: 35)
Isovaleric acid-DTHFPCIKFEPRS(K(Dapa-Palm))GCK-NH₂;
(Compound 36; SEQ ID NO: 36)
Isovaleric acid-DTHFPCIKFEPRSK(K(Dapa-Palm))CK-NH₂;
(Compound 37; SEQ ID NO: 37)
Isovaleric acid-DTHFPCIKFEPRSKGC(K(Dapa-Palm))K-NH₂;
(Compound 38; SEQ ID NO: 38)
Isovaleric acid-DTHFPCIKFEPRSKGC(K(Dapa-Palm))-NH₂;
(Compound 39; SEQ ID NO: 39)
Isovaleric acid-DTHFPCIKF(K(PEG11-Palm))PRSK[Sar]CK-NH₂;
(Compound 40; SEQ ID NO: 40)
Isolvaleric acid-DTHFPCIKF-NH₂;
(Compound 41; SEQ ID NO: 41)
Hy-DTHFPCIKF-NH₂;
(Compound 42; SEQ ID NO: 42)
Isolvaleric acid-DTHFPCIIF-NH₂;
(Compound 43; SEQ ID NO: 43)
Hy-DTHFPCIIKF-NH₂;
(Compound 44; SEQ ID NO: 44)
Isovaleric acid-DTKFPCIIF-NH₂;
(Compound 45; SEQ ID NO: 45)
Hy-DTKFPCIIF-NH₂;
and
(Compound 46; SEQ ID NO: 46)
Isovaleric_Acid-ETHFPCI(K(IsoGlu_Palm))FEPRSKGCK-NH₂
or a pharmaceutically acceptable salt thereof,
optionally, wherein the peptide has a disulfide bond formed between the thiol groups of two cysteine residues in the same peptide.

9. The method of claim 1, wherein the peptide is:
(Compound 2; SEQ ID NO: 2)
Isovaleric acid-DTHFPCIIFGPRSKGWVCK-NH₂;
or
(Compound 3; SEQ ID NO: 3)
Isovaleric acid-DTHFPCIIFEPRSKGWVCK-NH₂;
or
(Compound 7; SEQ ID NO: 7)
Isovaleric acid-DTHFPCI(K(PEG8))FGPRSKGWVCK-NH₂;
or
(Compound 8; SEQ ID NO: 8)
Isovaleric acid-DTHFPCIKF(K(PEG8))PRSKGWVCK-NH₂;
or
(Compound 11; SEQ ID NO: 9)
Isovaleric acid-DTHFPCIIFGPRSRGWVC(K(PEG8))-NH₂;
or
(Compound 14; SEQ ID NO: 14)
Isovaleric acid-DTHFPCI(K(Palm))FGPRSKGWVCK-NH₂;
or
(Compound 15; SEQ ID NO: 15)
Isovaleric acid-DTHFPCIKF(K(Palm))PRSKGWVCK-NH₂;
or
(Compound 16; SEQ ID NO: 16)
Isovaleric acid-DTHFPCIKFGP(K(Palm))SKGWVCK-NH₂;
or
(Compound 18; SEQ ID NO: 18)
Isovaleric acid-DTHFPCIKFGPRSKGWVC(K(Palm))-NH₂;
or
(Compound 19; SEQ ID NO: 19)
Isovaleric acid-DTHFPCI(K(PEG3-Palm))FGPRSKGWVCK-NH₂;
or
(Compound 20; SEQ ID NO: 20)
Isovaleric acid-DTHFPCIKF(K(PEG3-Palm))PRSKGWVCK-NH₂;
or
(Compound 21; SEQ ID NO: 21)
Isovaleric acid-DTHFPCIKFGP(K(PEG3-Palm))SKGWVCK-NH₂;
or
(Compound 22; SEQ ID NO: 22)
Isovaleric acid-DTHFPCIKFGPRS(K(PEG3-Palm))GWVCK-NH₂;
or
(Compound 23; SEQ ID NO: 23)
Isovaleric acid-DTHFPCIKFGPRSKGWVC(K(PEG3-Palm))-NH₂;
or
(Compound 24; SEQ ID NO: 24)
Isovaleric acid-DTHFPCIKFGPRSKGWVC(K(PEG8))-NH₂;
or
(Compound 25; SEQ ID NO: 25)
Isovaleric acid-DTHFPCI(K(isoGlu-Palm))FEPRSKGCK-NH₂;
or
(Compound 26; SEQ ID NO: 26)
Isovaleric acid-DTHFPCIKF(K(isoGlu-Palm))PRSKGCK-NH₂;
or
(Compound 27; SEQ ID NO: 27)
Isovaleric acid-DTHFPCIKFEP(K(isoGlu-Palm))SKGCK-NH₂;
or
(Compound 28; SEQ ID NO: 28)
Isovaleric acid-DTHFPCIKFEPRS(K(isoGlu-Palm))GCK-NH₂;
or
(Compound 32; SEQ ID NO: 32)
Isovaleric acid-DTHFPCI(K(Dapa-Palm))FEPRSKGCK-NH₂;
or
(Compound 34; SEQ ID NO: 34)
Isovaleric acid-DTHFPCIKFEP(K(Dapa-Palm))SKGCK-NH₂;
or
(Compound 46; SEQ ID NO: 46)
Isovaleric_Acid-ETHFPCI(K(IsoGlu_Palm))FEPRSKGCK-NH₂,
or a pharmaceutically acceptable salt thereof, wherein each peptide is optionally has a disulfide bond formed between the thiol groups of two cysteine residues in the peptide.

10. The method of claim 1, wherein the peptide is selected from: 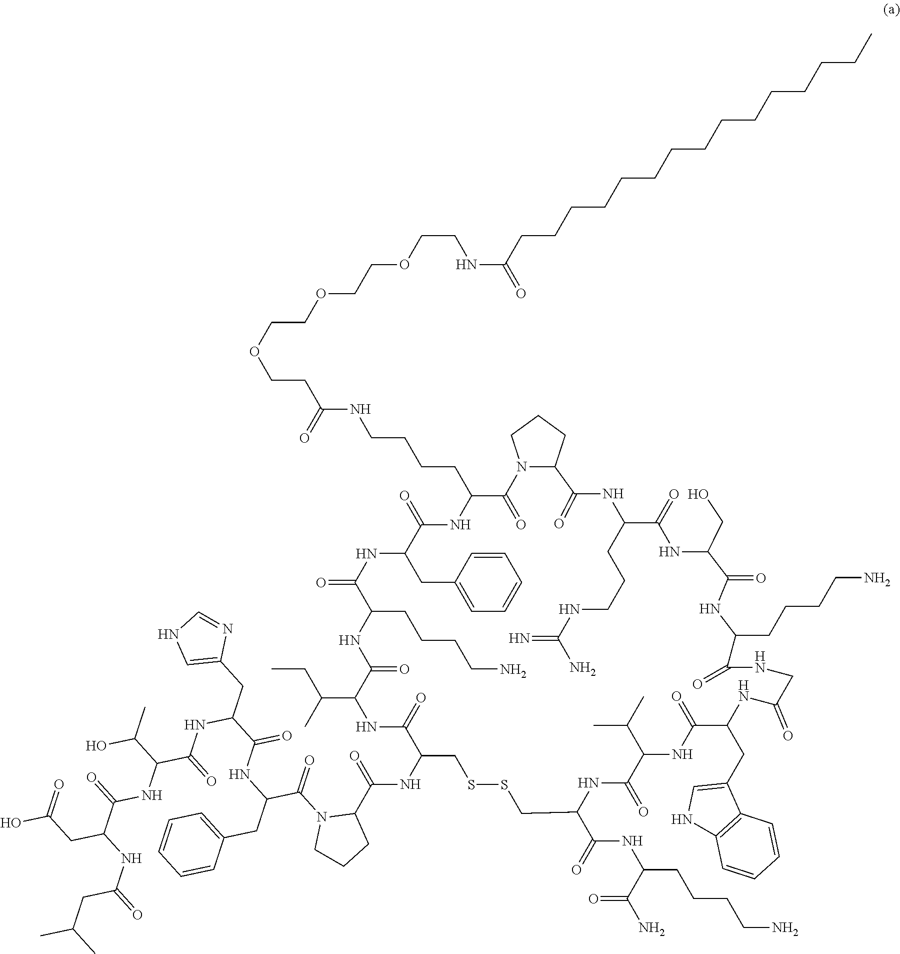 [SMILES: CCCCCCCCCCCCCCCC(=O)NCCOCCOCCOCCC(=O)NCCCCC1NC(=O)C(Cc2ccccc2)NC(=O)C(CCCCN)NC(=O)C(C(C)CC)NC(=O)C(NC(=O)C2CCCN2C(=O)C(Cc2ccccc2)NC(=O)C(Cc2c[nH]cn2)NC(=O)C(NC(=O)C(CC(=O)O)NC(=O)CC(C)C)C(C)O)CSSCC(C(=O)NC(CCCCN)C(N)=O)NC(=O)C(C(C)C)NC(=O)C(Cc2c[nH]c3ccccc23)NC(=O)CNC(=O)C(CCCCN)NC(=O)C(CO)NC(=O)C(CCCNC(=N)N)NC(=O)C2CCCN2C1=O]
Isovaleric acid-DTHFPCIKF(K(PEG3-Palm))PRSKGWVCK-NH₂ (Compound 20; SEQ ID NO:20); 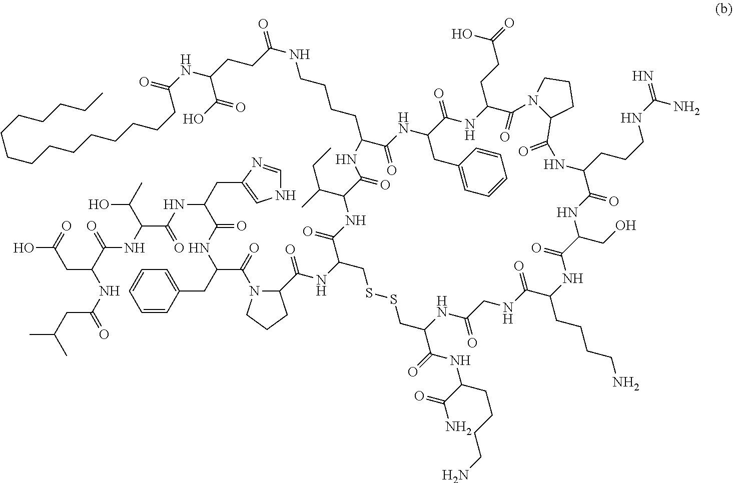 [SMILES: CCCCCCCCCCCCCCCC(=O)NC(CCC(=O)NCCCCC1NC(=O)C(C(C)CC)NC(=O)C(NC(=O)C2CCCN2C(=O)C(Cc2ccccc2)NC(=O)C(Cc2c[nH]cn2)NC(=O)C(NC(=O)C(CC(=O)O)NC(=O)CC(C)C)C(C)O)CSSCC(C(=O)NC(CCCCN)C(N)=O)NC(=O)CNC(=O)C(CCCCN)NC(=O)C(CO)NC(=O)C(CCCNC(=N)N)NC(=O)C2CCCN2C(=O)C(CCC(=O)O)NC(=O)C(Cc2ccccc2)NC1=O)C(=O)O]
Isovaleric acid-DTHFPCI(K(isoGlu-Palm))FEPRSKGCK-NH₂ (Compound 25; SEQ ID NO:25); 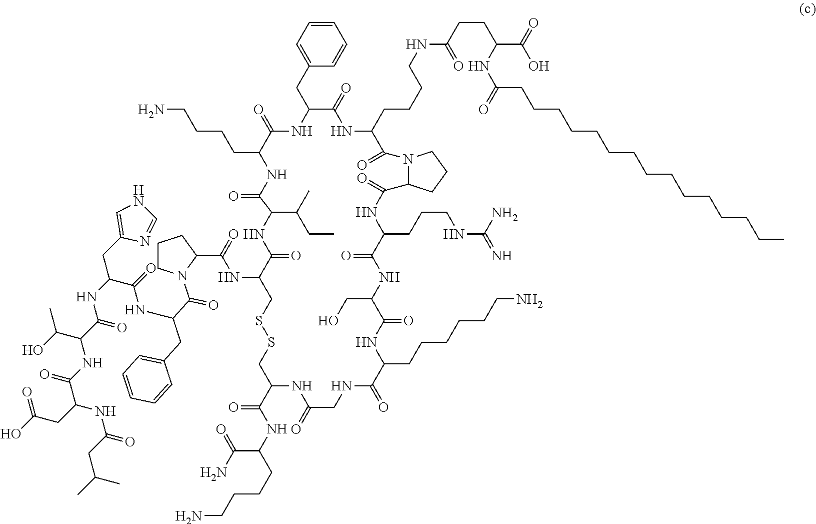 [SMILES: CCCCCCCCCCCCCCCC(=O)NC(CCC(=O)NCCCCC1NC(=O)C(Cc2ccccc2)NC(=O)C(CCCCN)NC(=O)C(C(C)CC)NC(=O)C(NC(=O)C2CCCN2C(=O)C(Cc2ccccc2)NC(=O)C(Cc2c[nH]cn2)NC(=O)C(NC(=O)C(CC(=O)O)NC(=O)CC(C)C)C(C)O)CSSCC(C(=O)NC(CCCCN)C(N)=O)NC(=O)CNC(=O)C(CCCCCCN)NC(=O)C(CO)NC(=O)C(CCCNC(=N)N)NC(=O)C2CCCN2C1=O)C(=O)O]
Isovaleric acid-DTHFPCIKF(K(isoGlu-Palm))PRSKGCK-NH₂ (Compound 26; SEQ ID NO:26); 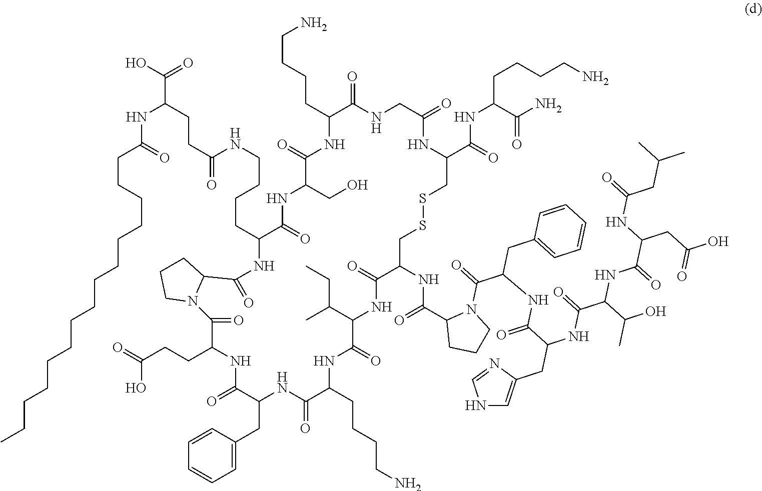 [SMILES: CCCCCCCCCCCCCCCC(=O)NC(CCC(=O)NCCCCC1NC(=O)C2CCCN2C(=O)C(CCC(=O)O)NC(=O)C(Cc2ccccc2)NC(=O)C(CCCCN)NC(=O)C(C(C)CC)NC(=O)C(NC(=O)C2CCCN2C(=O)C(Cc2ccccc2)NC(=O)C(Cc2c[nH]cn2)NC(=O)C(NC(=O)C(CC(=O)O)NC(=O)CC(C)C)C(C)O)CSSCC(C(=O)NC(CCCCN)C(N)=O)NC(=O)CNC(=O)C(CCCCN)NC(=O)C(CO)NC1=O)C(=O)O]
Isovaleric acid-DTIFPCIKFEP(K(isoGlu-Palm))SKGCK-NH₂ (Compound 27; SEQ TD NO:27); 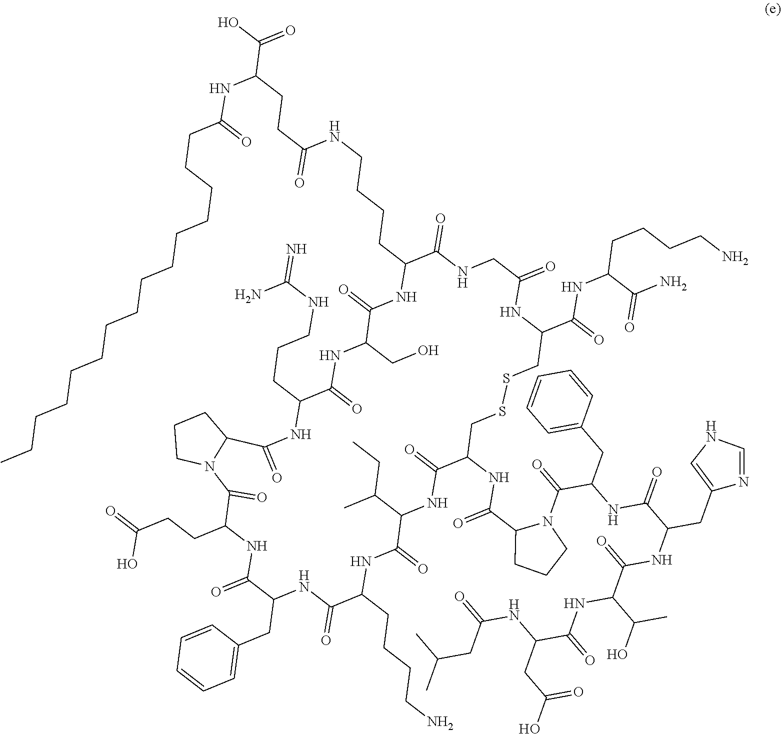 [SMILES: CCCCCCCCCCCCCCCC(=O)NC(CCC(=O)NCCCCC1NC(=O)C(CO)NC(=O)C(CCCNC(=N)N)NC(=O)C2CCCN2C(=O)C(CCC(=O)O)NC(=O)C(Cc2ccccc2)NC(=O)C(CCCCN)NC(=O)C(C(C)CC)NC(=O)C(NC(=O)C2CCCN2C(=O)C(Cc2ccccc2)NC(=O)C(Cc2c[nH]cn2)NC(=O)C(NC(=O)C(CC(=O)O)NC(=O)CC(C)C)C(C)O)CSSCC(C(=O)NC(CCCCN)C(N)=O)NC(=O)CNC1=O)C(=O)O]
Isovaleric acid-DTHFPCIKFEPRS(K(isoGlu-Palm))GCK-NH₂ (Compound 28; SEQ ID NO:28), 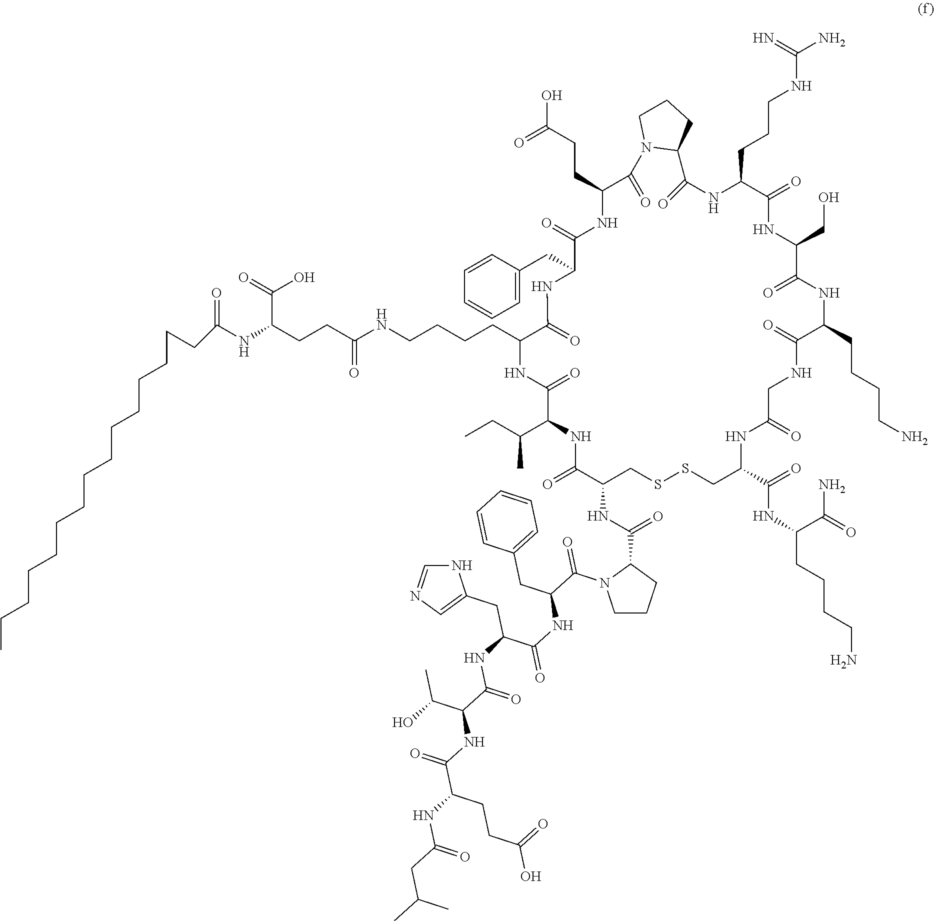 [SMILES: CCCCCCCCCCCCCCCC(=O)N[C@@H](CCC(=O)NCCCCC1NC(=O)[C@H]([C@@H](C)CC)NC(=O)[C@@H](NC(=O)[C@@H]2CCCN2C(=O)[C@H](Cc2ccccc2)NC(=O)[C@H](Cc2cnc[nH]2)NC(=O)[C@@H](NC(=O)[C@H](CCC(=O)O)NC(=O)CC(C)C)[C@@H](C)O)CSSC[C@@H](C(=O)N[C@@H](CCCCN)C(N)=O)NC(=O)CNC(=O)[C@H](CCCCN)NC(=O)[C@H](CO)NC(=O)[C@H](CCCNC(=N)N)NC(=O)[C@@H]2CCCN2C(=O)[C@H](CCC(=O)O)NC(=O)[C@H](Cc2ccccc2)NC1=O)C(=O)O]
Isovaleric_Acid-ETHFPCI(K(IsoGlu_Palm))FEPRSKGCK-NH₂ (Compound 46; SEQ ID NO:46), or a pharmaceutically acceptable salt thereof, wherein the amino acids are L-amino acids.

11. The method of claim 1, wherein the hepcidin mimetic or peptide is administered to the subject subcutaneously.

12. The method of claim 1, wherein the effective amount comprises a dose in the range of about 0.1 mg/kg body weight to about 100 mg/kg body weight, and optionally wherein the subject is administered different doses during different time periods over a course of treatment

13. The method of claim 1, wherein the subject is administered the effective amount of the hepcidin mimetic about once a week, about twice a week or about three times a week for at least some time period during the course of treatment.

14. The method of claim 1, wherein the hepcidin mimetic is a peptide having the formula: 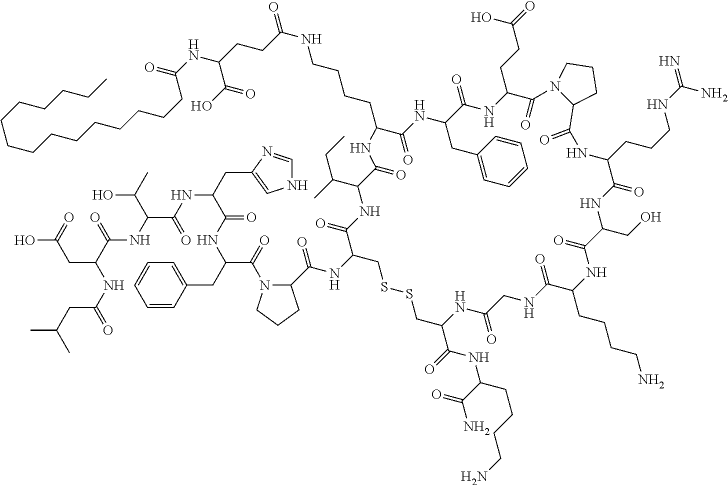 [SMILES: CCCCCCCCCCCCCCCC(=O)NC(CCC(=O)NCCCCC1NC(=O)C(C(C)CC)NC(=O)C(NC(=O)C2CCCN2C(=O)C(Cc2ccccc2)NC(=O)C(Cc2c[nH]cn2)NC(=O)C(NC(=O)C(CC(=O)O)NC(=O)CC(C)C)C(C)O)CSSCC(C(=O)NC(CCCCN)C(N)=O)NC(=O)CNC(=O)C(CCCCN)NC(=O)C(CO)NC(=O)C(CCCNC(=N)N)NC(=O)C2CCCN2C(=O)C(CCC(=O)O)NC(=O)C(Cc2ccccc2)NC1=O)C(=O)O]
Isovaleric acid-DTTIFPCI(K(isoGlu-Palm))FEPRSKGCK-NH₂ (Compound 25; SEQ TD NO:25), or a pharmaceutically acceptable salt thereof,

15. The method of claim 1, wherein the hepcidin mimetic is a peptide having the formula: 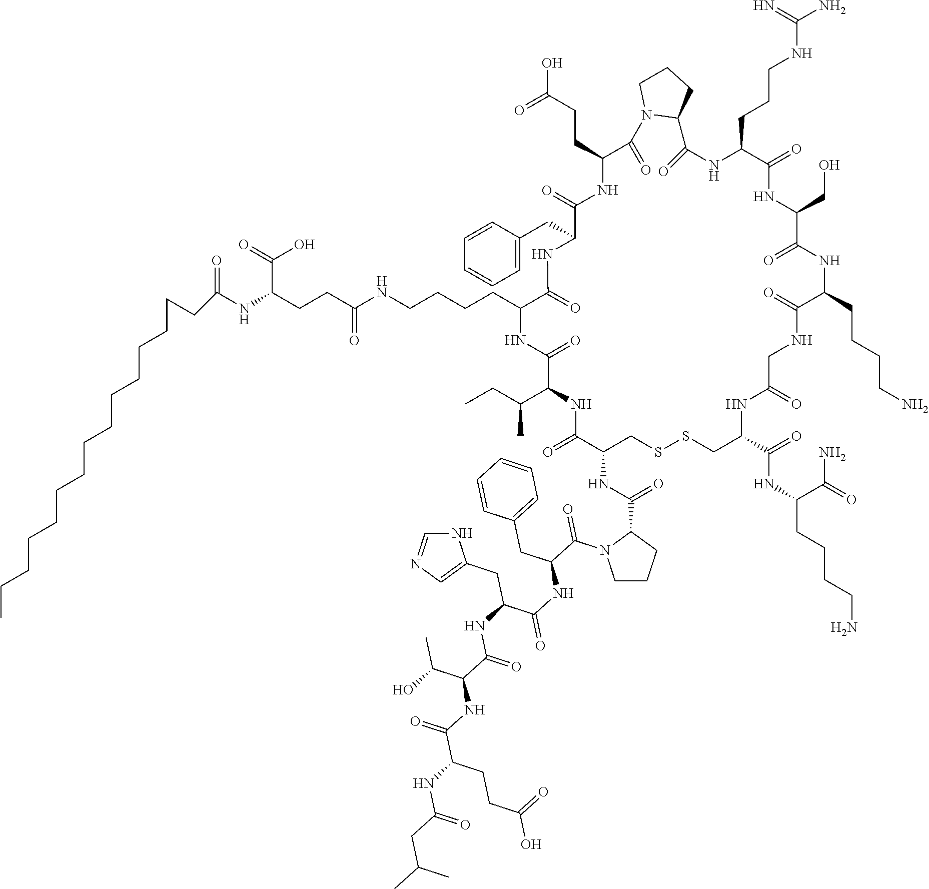 [SMILES: CCCCCCCCCCCCCCCC(=O)N[C@@H](CCC(=O)NCCCCC1NC(=O)[C@H]([C@@H](C)CC)NC(=O)[C@@H](NC(=O)[C@@H]2CCCN2C(=O)[C@H](Cc2ccccc2)NC(=O)[C@H](Cc2cnc[nH]2)NC(=O)[C@@H](NC(=O)[C@H](CCC(=O)O)NC(=O)CC(C)C)[C@@H](C)O)CSSC[C@@H](C(=O)N[C@@H](CCCCN)C(N)=O)NC(=O)CNC(=O)[C@H](CCCCN)NC(=O)[C@H](CO)NC(=O)[C@H](CCCNC(=N)N)NC(=O)[C@@H]2CCCN2C(=O)[C@H](CCC(=O)O)NC(=O)[C@H](Cc2ccccc2)NC1=O)C(=O)O]
Isovaleric_Acid-ETHFPCI(K(IsoGlu_Palm))FEPRSKGCK-NH₂ (Compound 46; SEQ ID NO:46), or a pharmaceutically acceptable salt thereof.

16. The method of claim 1, wherein the sickle cell disease is sickle cell anemia (HbSS), HbSβ0 thalassemia, HbSβ+thalassemia or hemoglobin SC disease (HbSC).

17. The method of claim 1, wherein the subject has a reduction in red blood cell counts.

18. The method of claim 1, wherein the subject has a reduction in hemoglobin counts.

19. The method of claim 1, wherein the subject has a reduction in hematocrit.

20. The method of claim 1, wherein the subject has a reduction in lymphocyte counts.
